(19) United States
(12) Patent Application Publication
(10) Pub. No.: US 2023/0407255 A1
(43) Pub. Date: Dec. 21, 2023

(54) DENDRITIC CELL ASSAY FOR INNATE IMMUNOGENICITY TO GENE THERAPY AGENTS

(71) Applicant: Genzyme Corporation, Cambridge, MA (US)

(72) Inventors: Sourav Roy CHOUDHURY, Waban, MA (US); Mona MOTWANI, Framingham, MA (US)

(21) Appl. No.: 18/299,656

(22) Filed: Apr. 12, 2023

Related U.S. Application Data
(60) Provisional application No. 63/330,241, filed on Apr. 12, 2022.

(51) Int. Cl. C12N 5/0786; C12N 15/86; A61K 48/00

(52) U.S. Cl. CPC C12N 5/0645; C12N 15/86; A61K 48/005; C12N 2502/1121; C12N 2750/14143

(57) ABSTRACT

Provided herein are methods for determining the innate immunogenicity of a gene therapy agent in an individual. In particular, the methods involve the use of isolated dendritic cells to detect innate immunogenicity to a gene therapy agent. Exemplary gene therapy agents include adeno-associated virus (AAV) vectors, adenovirus vectors, lentivirus vectors, Herpes simplex virus (HSV) vectors or a lipid nanoparticles.

### CROSS REFERENCE TO RELATED APPLICATIONS

This application claims the priority benefit of U.S. Provisional Application No. 63/330,241, filed Apr. 12, 2022, which is incorporated by reference in its entirety.

### REFERENCE TO AN ELECTRONIC SEQUENCE LISTING

The contents of the electronic sequence listing (159792018100SEQLIST.xml; Size: 2,016 bytes; and Date of Creation: Apr. 12, 2023) is herein incorporated by reference in its entirety.

### FIELD OF THE INVENTION

The present invention relates to methods for determining innate immunogenicity to a gene therapy agent using dendritic cells.

### BACKGROUND OF THE INVENTION

Current success of gene therapy for treatment of rare genetic diseases relies heavily on adeno-associated viral (AAV) vectors, which provide several attractive features including tissue specific tropism, transduction of quiescent cells and long-term persistence of transgene expression. However, immune responses to AAV vectors pose a major challenge for successful clinical translation. All components of the viral vector—capsid, viral genome as well as transgene—trigger immune responses that involve activation of both innate and adaptive arms of human immune system. The adaptive immune responses to AAV triggered by B and T cells have been relatively well characterized; however, innate immune activation by AAV is very poorly understood. A major challenge in our understanding of innate immune response to AAV lies in the poor reproducibility of immune signaling observed in clinical trials, to an ex vivo setting. What is needed is a novel assay that recapitulates innate immune signature in several human donors in response to a variety of AAV serotypes.

All references cited herein, including patent applications and publications, are incorporated by reference in their entirety.

### BRIEF SUMMARY OF THE INVENTION

In some aspects, the invention provides methods of determining the innate immunogenicity to a gene therapy agent in an individual, the method comprising a) incubating an innate immune cell from the individual with the gene therapy agent, b) analyzing the innate immune cell for altered expression of one or more cytokines compared to a suitable control, whereby altered expression of the one or more cytokines produces a cytokine signature, wherein expression of a cytokine signature following incubation with the gene therapy agent indicates innate immunogenicity to the gene therapy agent in the individual. In some embodiments, the innate immune cell is a dendritic cell, a monocyte, a macrophage or a natural killer (NK) cell. In some embodiments, the innate immune cell is isolated from peripheral blood mononuclear cells from the individual. In some embodiments, the innate immune cell is a dendritic cell. In some embodiments, the dendritic cell is derived from a monocyte of the individual.

In some embodiments of the invention, the method further comprises isolating monocytes from the individual and incubating the monocytes in dendritic cell culture media to derive dendritic cells from the monocytes prior to incubating the dendritic cells with the gene therapy agent. In some embodiments, the monocytes are CD14+ monocytes. In some embodiments, the monocytes are incubated with the dendritic cell culture media for about 5 to about 10 days or about 7 to about 8 days to derive dendritic cells from the monocytes. In some embodiments, the innate immune cells are replated prior to the incubation with the gene therapy agent of step b). In some embodiments, the innate immune cells are replated into microwell dishes.

In some embodiments of the invention, the gene therapy agent is a viral vector and wherein the innate immune cells are incubated with the gene therapy agent at an MOI of about 1×10³ to about 1×10⁵ or about 1×10⁴. In some embodiments, the gene therapy agent is a non-viral vector, and wherein the innate immune cells are incubated with the non-viral vector at a concentration of about 1 ng/mL to about 1 mg/mL. In some embodiments, the innate immune cells are incubated with the gene therapy agent for about 12 hours to about 36 hours or about 24 hours.

In some embodiments, the cytokine signature comprises increased expression of IL6, TNFα, and IL-1β. In some embodiments, the cytokine signature comprises increased expression of one or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β. In some embodiments, expression of the cytokines in the cytokine signature is increased compared to a suitable control. In some embodiments, the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

In some aspects, the invention provides methods of determining the innate immunogenicity to a viral gene therapy agent in an individual, the method comprising a) incubating monocytes from the individual in dendritic cell culture media under conditions in which the monocytes differentiate into dendritic cells, b) incubating the dendritic cells with the viral gene therapy agent at an MOI of about 1×10³ to about 1×10⁵ for about 12 to about 36 hours, c) analyzing the dendritic cells for altered expression of one or more cytokines compared to a suitable control, whereby altered expression of the one or more cytokines produces a cytokine signature, wherein expression of the cytokine signature following incubation with the viral gene therapy agent indicates innate immunogenicity to the viral gene therapy agent in the individual, wherein the cytokine signature comprises increased expression of IL6, TNFα, and IL-1β. In some embodiments, the monocytes are obtained from peripheral mononuclear cells from the individual. In some embodiments, the monocytes are CD14+ monocytes. In some embodiments, the monocytes are incubated in dendritic cell culture media for about 7-8 days to differentiate the monocytes to dendritic cells. In some embodiments, the dendritic cells are incubated with the viral gene therapy agent at an MOI of about 1×10⁴. In some embodiments, the dendritic cells are incubated with the viral gene therapy agent for about 24 hours.

In some embodiments of the invention, the viral vector is an AAV particle. In some embodiments, the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LKO3 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof. In some embodiments, the AAV capsid comprises a tyrosine mutation, a heparin binding mutation, or an HBKO mutation. In some embodiments, the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR. In some embodiments, the one or more ITRs and the capsid of the AAV particle are derived from the same AAV serotype. In some embodiments, the one or more ITRs and the capsid of the AAV particles are derived from different AAV serotypes.

In some embodiments of the invention, the viral vector is an adenoviral particle. In some embodiments, the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

In some embodiments of the invention, the viral vector is a lentiviral particle. In some embodiments, the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

In some embodiments of the invention, the viral vector is a Herpes simplex virus (HSV) particle. In some embodiments, the HSV particle is an HSV-1 particle or an HSV-2 particle, or a functional variant thereof.

In some aspects, the invention provides methods of determining the innate immunogenicity to a non-viral gene therapy agent in an individual, the method comprising a) incubating monocytes from the individual in dendritic cell culture media under conditions in which the monocytes differentiate into dendritic cells, b) incubating the dendritic cells with the non-viral vector at a concentration of about 1 ng/mL to about 1 mg/mL, c) analyzing the dendritic cells for altered expression of one or more cytokines compared to a suitable control, whereby altered expression of the one or more cytokines produces a cytokine signature, wherein expression of the cytokine signature following incubation with the non-viral gene therapy agent indicates innate immunogenicity to the non-viral gene therapy agent in the individual, wherein the cytokine signature comprises increased expression of IL6, TNFα, and IL-1β. In some embodiments, the monocytes are obtained from peripheral mononuclear cells from the individual. In some embodiments, the monocytes are CD14+ monocytes. In some embodiments, the monocytes are incubated in dendritic cell culture media for about 7-8 days to differentiate the monocytes to dendritic cells. In some embodiments, the dendritic cells are incubated with the non-viral gene therapy agent for about 12 hours to about 36 hours or about 24 hours.

In some embodiments, the gene therapy agent comprises nucleic acid encoding a heterologous transgene. In some embodiments, the heterologous transgene is operably linked to a promoter. In some embodiments, the promoter is a constitutive promoter, a tissue-specific promoter, or an inducible promoter.

In some aspects, the invention provides methods of determining a cytokine signature of a gene therapy agent comprising a) incubating one or more innate immune cell from one or more individuals with the gene therapy agent, b) analyzing the one or more innate immune cells for altered expression of one or more cytokines compared to a suitable control, wherein the altered expression of one or more cytokines in step b) indicates the cytokine signature of the gene therapy agent. In some embodiments, the innate immune cell is a dendritic cell, a monocyte, a macrophage or a natural killer (NK) cell. In some embodiments, the one or more innate immune cells are isolated from peripheral blood mononuclear cells from the individual. In some embodiments, the innate immune cell is a dendritic cell. In some embodiments, the dendritic cell is derived from a monocyte of the one or more individuals.

In some embodiments of the invention, the methods further comprise isolating monocytes from the one or more individuals and incubating the monocytes in dendritic cell culture media to derive dendritic cells from the monocytes prior to incubating the dendritic cells with the gene therapy agent. In some embodiments, the monocytes are CD14+ monocytes. In some embodiments, the monocytes are incubated with the dendritic cell culture media for about 5 to about 10 days or about 7 to about 8 days to derive dendritic cells from the monocytes. In some embodiments, the innate immune cells are replated prior to the incubation with the gene therapy agent of step b).

In some embodiments, the gene therapy agent is a viral vector and wherein the innate immune cells are incubated with the gene therapy agent at an MOI of about 1×10³ to about 1×10⁵ or about 1×10⁴. In some embodiments, the gene therapy agent is a non-viral vector, and wherein the innate immune cells are incubated with the non-viral vector at a concentration of about 1 ng/mL to about 1 mg/mL. In some embodiments, the innate immune cells are incubated with the gene therapy agent for about 12 hours to about 36 hours or about 24 hours.

In some embodiments of the invention, expression of the cytokines in the cytokine signature is increased compared to a suitable control. In some embodiments, the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent. In some embodiments, the gene therapy agent is a viral vector or a non-viral vector.

In some embodiments, the viral vector is an AAV particle. In some embodiments, the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LKO3 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof. In some embodiments, the AAV capsid comprises a tyrosine mutation, a heparin binding mutation, or an HBKO mutation. In some embodiments, the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR. In some embodiments, the one or more ITRs and the capsid of the AAV particle are derived from the same AAV serotype. In some embodiments, the one or more ITRs and the capsid of the AAV particles are derived from different AAV serotypes.

In some embodiments, the viral vector is an adenoviral particle. In some embodiments, the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

In some embodiments, the viral vector is a lentiviral particle. In some embodiments, the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

In some embodiments, the viral vector is a Herpes simplex virus (HSV) particle. In some embodiments, the HSV particle is an HSV-1 particle or an HSV-2 particle, or a functional variant thereof.

In some embodiments, the gene therapy agent comprises nucleic acid encoding a heterologous transgene. In some embodiments, the heterologous transgene is operably linked to a promoter. In some embodiments, the promoter is a constitutive promoter, a tissue-specific promoter, or an inducible promoter.

In some embodiments, the invention provides a kit for use in any of the methods of the invention.

### BRIEF DESCRIPTION OF THE DRAWINGS

FIG. 1 shows a schematic of the assay to determine innate immunogenicity of different AAV vectors.

FIG. 2 shows cytokine levels from AAV treated cells as determined by Luminex assay. AAV serotype is indicated above each plot; cytokines assayed are shown on the left; donor IDs are shown along the top of each plot. Colors within each plot correspond to a change in regulation as compared to uninfected cells and indicated in the bottom right panel.

FIG. 3 shows cytokine levels from AAV treated cells as determined by an orthogonal MSD assay. AAV serotype is indicated above each plot; cytokines assayed are shown on the left; donor IDs are shown along the top of each plot. Colors within each plot correspond to a change in regulation as compared to uninfected cells and indicated in the bottom right panel.

FIG. 4 shows an experimental schematic for assaying the innate immune response following LNP delivery. Peripheral blood monocuclear cells (PBMCs) were isolated from leukopaks. CD14+ monocytes were purified from (PBMCs). Differentiation factor cocktail was added to the monocytes to allow differentiation to dendritic cells and finally maturation factors were added to obtain mature dendritic cells. The mature dendritic cells were treated with 10 ug of LNPs for 24 hrs. The cells were then harvested for measuring cellular toxicity and target gene expression using flow cytometry and media was collected for assessment of cytokine release.

FIG. 5 shows that the dendritic cell system of the disclosure can be used to assess immunogenicity of LNPs without impacting cell viability.

FIG. 6 shows that the dendritic cell system of the disclosure can be used to assess LNP transduction effectively. Specially, dendritic cells produced in accordance with the disclosure were effectively transduced by LNPs encapsulating mRNA.

FIGS. 7A-7D show that mRNAs encapsulating LNPs specifically cytokines as compared to cells that had no LNP treatment. FIG. 7A shows the results for IP10 secretion.

FIG. 7B shows the results for MIP1b secretion. FIG. 7C shows the results for CXCL9 secretion. FIG. 7D shows the results for IL2 secretion.

### DETAILED DESCRIPTION

In some aspects, the invention provides methods of determining the innate immunogenicity to a gene therapy agent in an individual, the method comprising a) incubating an innate immune cell from the individual with the gene therapy agent, b) analyzing the innate immune cell for altered expression of one or more cytokines compared to a suitable control, whereby altered expression of the one or more cytokines produces a cytokine signature, wherein expression of a cytokine signature following incubation with the gene therapy agent indicates innate immunogenicity to the gene therapy agent in the individual. In some embodiments, the innate immune cell is a dendritic cell, a monocyte, a macrophage, or a natural killer (NK) cell. In some embodiments, the method further comprises isolating monocytes from the individual and incubating the monocytes in dendritic cell culture media to derive dendritic cells from the monocytes prior to incubating the dendritic cells with the gene therapy agent. In some embodiments, the cytokine signature comprises one or more of IL6, TNFα, IL-1β, MCP1 and MIP-1α. In some embodiments, the method of determining whether an individual has innate immunity to a gene therapy agent is determined prior to administration of the gene therapy agent to the individual. In some embodiments, the method of determining whether an individual has innate immunity to a gene therapy agent is determined prior to administration of the gene therapy agent to the individual, thereby identifying an individual that would benefit from administration of a modulator of the innate immune response either before, during or after administration of the gene therapy agent.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer. Principles and Practice of Oncology (V. T. DeVita et al., eds., J.B. Lippincott Company, 2011).

### Definitions

As used herein, the term “IRAK degrader” is a heterobifunctional compound that binds to and/or inhibits (completely or partially) both an IRAK kinase and an E3 ligase with measurable affinity resulting in the ubiqitination and subsequent degradation of the IRAK kinase. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

As used herein, the term “inhibitor” in reference to IRAK is a compound that binds to and/or inhibits (completely or partially) an IRAK kinase with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “modulator” in reference to IRAK is a compound that stimulates, delays, inhibits and/or or suppresses (completely or partially) the activity of an IRAK kinase

As used herein, the term “gene therapy” refers to a therapy whereby the expression of a nucleic acid (e.g., a gene, an mRNA, etc.) in a cell of an individual is modified to alter the biological properties of the cell. In some examples, the gene therapy includes delivery of exogenous nucleic acid to be expressed in a cell in an individual. In some examples, the gene therapy alters (e.g., degrades, inhibits, enhances) the expression of an endogenous gene in a cell of an individual. In some examples, the gene therapy is an in vivo therapy. In some examples, the gene therapy is an ex vivo therapy (e.g., a cell therapy).

As used herein, the term “gene therapy agent” refers to a nucleic acid (e.g., expression construct, miRNA, antisense, shRNA, siRNA) or a nucleic acid in combination with an agent used to deliver the nucleic acid to an individual or a cell to modify or manipulate the expression of one or more nucleic acids (e.g., gene, mRNA) in an individual or a cell to alter the biological propertied of living cells. Examples of gene therapy agents include, but are not limited to, viral vectors (e.g., adeno-associated virus, adenovirus, lentivirus, Herpes simples virus, baculovirus), bacterial vectors, and non-viral vectors (e.g., lipid nanoparticles (LNPs) encapsulating a therapeutic nucleic acid or plasmid DNAs (e.g., close endedDNA) comprising a therapeutic nucleic acid and/or encoding a therapeutic polypeptide).

A “vector,” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups. Alternatively, the backbone of the nucleic acid can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded nucleic acid can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one, e.g., two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and, in embodiments, encapsidated in a viral particle, particularly an AAV particle. A rAAV vector can be packaged into an AAV virus capsid to generate a “recombinant adeno-associated viral particle (rAAV particle)”.

An “rAAV virus” or “rAAV viral particle” refers to a viral particle composed of at least one AAV capsid protein and an encapsidated rAAV vector genome.

A “recombinant adenoviral vector” refers to a polynucleotide vector comprising one or more heterologous sequences (i.e., nucleic acid sequence not of adenovirus origin) that are flanked by at least one adenovirus inverted terminal repeat sequence (ITR). In some embodiments, the recombinant nucleic acid is flanked by two inverted terminal repeat sequences (ITRs). Such recombinant viral vectors can be replicated and packaged into infectious viral particles when present in a host cell that is expressing essential adenovirus genes deleted from the recombinant viral genome (e.g., E1 genes, E2 genes, E4 genes, etc.). When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of adenovirus packaging functions. A recombinant viral vector can be in any of a number of forms, including, but not limited to, plasmids, linear artificial chromosomes, complexed with lipids, encapsulated within liposomes, and encapsidated in a viral particle, for example, an adenovirus particle. A recombinant viral vector can be packaged into an adenovirus virus capsid to generate a “recombinant adenoviral particle.”

A “recombinant lentivirus vector” refers to a polynucleotide vector comprising one or more heterologous sequences (i.e., nucleic acid sequence not of lentivirus origin) that are flanked by at least one lentivirus terminal repeat sequences (LTRs). In some embodiments, the recombinant nucleic acid is flanked by two lentiviral terminal repeat sequences (LTRs). Such recombinant viral vectors can be replicated and packaged into infectious viral particles when present in a host cell that has been infected with a suitable helper functions. A recombinant lentiviral vector can be packaged into a lentivirus capsid to generate a “recombinant lentiviral particle.”

A “recombinant herpes simplex vector (recombinant HSV vector)” refers to a polynucleotide vector comprising one or more heterologous sequences (i.e., nucleic acid sequence not of HSV origin) that are flanked by HSV terminal repeat sequences. Such recombinant viral vectors can be replicated and packaged into infectious viral particles when present in a host cell that has been infected with a suitable helper functions. When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of HSV packaging functions. A recombinant viral vector can be in any of a number of forms, including, but not limited to, plasmids, linear artificial chromosomes, complexed with lipids, encapsulated within liposomes, and encapsidated in a viral particle, for example, an HSV particle. A recombinant viral vector can be packaged into an HSV capsid to generate a “recombinant herpes simplex viral particle.”

“Solid lipid nanoparticles” (SLNs, sLNPs), or “lipid nanoparticles” (LNPs) as used herein refer to nanoparticles composed of lipids. In some examples, there is only one phospholipid layer and the bulk of the interior of the particle is composed of lipophilic substance. Payloads such as nucleic acids can be embedded in the interior. In some examples, the lipid nanoparticle is a liposome, which comprise a lipid bilayer.

As used herein, the term “improving” as it relates to gene therapy may refer to the act of boosting, heightening, lengthening or otherwise increasing the expression of the therapeutic gene payload of a gene therapy agent. In some embodiments, an improved gene therapy is one where expression of the therapeutic gene payload of the gene therapy agent administered with an IRAK modulator is increased by greater than any of about 10%, 25%, 50%, 75%, or 100% compared to gene therapy administered without the IRAK modulator. In some embodiments, an improved gene therapy is one where time of expression of the therapeutic gene payload of the gene therapy agent administered with an IRAK modulator is lengthened by greater than any of about 10%, 25%, 50%, 75%, or 100% compared to gene therapy administered without the IRAK modulator. In some examples, a gene therapy is improved by decreasing an immune response (e.g., an innate immune response) to the gene therapy agent. In some embodiments, an improved gene therapy is one where an immune response to gene therapy agent administered with an IRAK modulator is decreased by greater than any of about 10%, 25%, 50%, 75%, or 100% compared to gene therapy administered without the IRAK modulator. In some embodiments, the decrease in an immune response to a gene therapy agent is measured as a decrease in a cytokine signature following exposure of the gene therapy agent to immune cells in the presence of an IRAK modulator compared to exposure of the gene therapy agent to immune cells in the absence of an IRAK modulator.

As used herein, the term “modulating” as it refers to gene therapy may refer to the act of changing, altering, varying, improving or otherwise modifying the presence, or an activity of, a gene therapy agent. For example, modulating an immune response to a gene therapy agent may refer to any act leading to changing, altering, varying, improving or otherwise modifying an immune response to the gene therapy agent (e.g., decreasing, delaying and/or eliminating an immune response (e.g., an innate immune response) to the gene therapy agent).

As used herein, the term “cytokine signature” as it relates to an immune response (e.g., innate immune response) to a gene therapy agent refers to altered (e.g., increased, decreased) expression of one or more cytokines following exposure of an innate immune cell to a gene therapy agent. In some examples, the cytokines of the cytokine signature are specific to a TLR pathway (e.g., a TLR2, TLR3, TLR4 or TLR9 pathway).

Innate immune cells are white blood cells that mediate innate immunity and include basophils, dendritic cells, eosinophils, Langerhans cells, mast cells, monocytes and macrophages, neutrophils and NK cells. Different AAV capsids can enter these innate immune cells with different efficiencies often referred to as transduction efficiency. Some serotypes such as AAV1 are efficient at transducing certain immune cells like monocytes whereas other AAVs like AAV6 are efficient at transducing cells like dendritic cells (Grimm, D et al., J. Virol., 2008, 82(12):5887-5911). However, all AAVs upon cell entry evoke immune response. The magnitude of this immune response is dependent on AAV serotype and cell type. Once AAVs transduce a host immune cell they engage immune receptors such as TLRs namely TLR9. Several studies using mouse models reveal that TLR9 is a key DNA sensor contributing to AAV immunogenicity (Zhu, J et al., J Clin Invest. 2009; 119(8):2388-2398; Ashley S N et al., Cell. Immunol. 2019, 346:103997). Once these TLRs are activated by viruses they secrete cytokines that establish an anti-viral state within the infected cell and alert the neighboring cells. (Carty, M and Bowie, AG, Clin Exp Immunol, 2010, 161(3):397-406; Lester, SN and Li, K, J Mol Biol. 2014; 426(6):1246-1264; Fitzgerald, K A and Kagan, J C, Cell, 2020 180(6):1044-1066).

These cytokines are also responsible for activating the adaptive immune system that comprises B cells and T cells which produce antibodies and generate cytotoxicity to kill the viral infected cells respectively. As used herein, the upregulation or downregulation of certain subset of cytokines is referred to as a “cytokine signature”. These cytokine signatures comprising one or more (e.g., three or more) cytokines can be used as predictive markers for diseases and success of therapies. Examples of cytokine signatures are found in Zuniga, J et al., Int. J. Infect. Diseases, 2020, 94:4-11, Bergamaschi, C et al., Cell Reports, 2021, 36:109504; Del Valle, D M et al., Nat. Med. 2020, 26:1636-1643.

“Heterologous” means derived from a genotypically distinct entity from that of the rest of the entity to which it is compared or into which it is introduced or incorporated. For example, a nucleic acid introduced by genetic engineering techniques into a different cell type is a heterologous nucleic acid (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a nucleic acid that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as siRNA.

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Viral., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Viral., 70:520-532 (LFU assay).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins. “AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A number of such helper viruses have been identified, including adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

“Percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. A potential alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

An “effective amount” of an agent refers to an amount effective, at dosages and for periods of time necessary, to achieve the desired therapeutic result. For example, an effective amount of a gene therapy agent refers to an amount effective, at dosages and for periods of time necessary, to achieve the desired gene therapeutic result. In another example, an effective amount of an IRAK modulator may refer to an amount effective, at dosages and for periods of time necessary, to achieve the desired result of improved gene therapy.

A “therapeutically effective amount” of a substance/molecule of the invention, (e.g., a gene therapy agent and/or an IRAK modulator) may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule are outweighed by the therapeutically beneficial effects.

The term “suitable control” as it refers to a cytokine signature is the expression of the cytokines in the cytokine signature from innate immune cells that are not incubated with the gene therapy agent or the expression of the cytokines in the cytokine signature from innate immune cells prior to incubation with the gene therapy agent.

Administration “in combination with” as it related to a gene therapy agent and a modulator of an innate immune response (e.g., an IRAK modulator) includes simultaneous (concurrent), consecutive or sequential administration in any order of the gene therapy agent and the modulator of an innate immune response (e.g., an IRAK modulator).

The term “concurrently” is used herein to refer to administration of a gene therapy agent and a modulator of an innate immune response (e.g., an IRAK modulator), where at least part of the administration overlaps in time. Accordingly, concurrent administration includes a dosing regimen when the administration of a gene therapy agent or a modulator of an innate immune response (e.g., an IRAK modulator) continues after discontinuing the administration of the other agent/modulator.

As used herein, “in conjunction with” refers to administration of one treatment modality in addition to another treatment modality. As such, “in conjunction with” refers to administration of one treatment modality (a gene therapy agent or a modulator of an innate immune response (e.g., an IRAK modulator)) before, during or after administration of the other treatment modality to the individual.

An “isolated” molecule (e.g., nucleic acid or protein) or cell means it has been identified and separated and/or recovered from a component of its natural environment.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising,” “consisting,” and/or “consisting essentially of” aspects and embodiments.

### Cell Assay for Determining Innate Immunogenicity to Gene Therapy Agents

In some aspects, the invention provides method for determining the innate immunogenicity to a gene therapy agent in an individual, wherein the innate immunogenicity can be directed toward any aspect of the gene therapy agent; for example, the delivery vehicle (e.g., a viral capsid or lipid nanoparticle), the nucleic acid payload of the gene therapy agent and/or any other components of the gene therapy agent.

In some aspects, the invention provides methods for determining the innate immunogenicity to a gene therapy agent in an individual, the method comprising a) incubating innate immune cells from the individual with the gene therapy agent, b) analyzing the innate immune cells for the expression of one or more cytokines wherein expression of a cytokine signature following incubation with the gene therapy agent indicates innate immunogenicity to the gene therapy agent. In some embodiments, the innate immune cell is a dendritic cell, a monocyte, a macrophage or a natural killer (NK) cell. In some embodiments, the innate immune cell is isolated from blood (e.g., peripheral blood mononuclear cells) from the individual. In some embodiments, the innate immune cell is a dendritic cell. In some embodiments, the dendritic cell is derived from a monocyte (e.g., a CD14+ monocyte) of the individual. As used herein, the term to “derive” dendritic cells includes differentiation of cells (e.g., monocytes) to produce dendritic cells. In some embodiments, the method further comprises isolating monocytes from the individual and incubating the monocytes in dendritic cell culture media under conditions in which dendritic cells are derived from (differentiated from) the monocytes prior to incubating the dendritic cells with the gene therapy agent.

In some aspects, the invention provides methods for determining the innate immunogenicity of a gene therapy agent in an individual, the method comprising a) incubating dendritic cells from the individual with the gene therapy agent, b) analyzing the dendritic cells for the expression of one or more cytokines wherein expression of a cytokine signature following incubation with the gene therapy agent indicates innate immunogenicity to the gene therapy agent. In some embodiments, the dendritic cell is isolated from peripheral blood mononuclear cells from the individual. In some embodiments, the dendritic cell is derived from a monocyte (e.g., a CD14+ monocyte) of the individual. In some embodiments, the method further comprises isolating monocytes from the individual and incubating the monocytes in dendritic cell culture media to derive dendritic cells from the monocytes prior to incubating the dendritic cells with the gene therapy agent. In some embodiments, the cytokine signature comprises increased expression of IL6, TNFα, and IL-1β.

In some aspects, the invention provides methods for determining the innate immunogenicity to a gene therapy agent in an individual, the method comprising a) isolating innate immune cells from the individual b) incubating the innate immune cells with the gene therapy agent, c) analyzing the innate immune cells for the expression of one or more cytokines wherein expression of a cytokine signature following incubation with the gene therapy agent indicates innate immunogenicity to the gene therapy agent. In some embodiments, the innate immune cell is a dendritic cell, a monocyte, a macrophage or a natural killer (NK) cell. In some embodiments, the innate immune cell is isolated from peripheral blood mononuclear cells from the individual. In some embodiments, the innate immune cell is a dendritic cell. In some embodiments, the dendritic cell is derived from a monocyte (e.g., a CD14+ monocyte) of the individual.

In some aspects, the invention provides methods for determining the innate immunogenicity of a gene therapy agent in an individual, the method comprising a) isolating monocytes from the individual b) incubating the monocytes in dendritic cell culture media to derive dendritic cells from the monocytes, c) incubating the dendritic cells with the gene therapy agent, d) analyzing the dendritic cells for the expression of one or more cytokines wherein expression of a cytokine signature following incubation with the gene therapy agent indicates innate immunogenicity to the gene therapy agent. In some embodiments, the cytokine signature comprises increased expression of IL6, TNFα, and IL-1β.

In some aspects, the invention provides methods for determining the innate immunogenicity to a viral gene therapy agent in an individual, the method comprising: a) incubating innate immune cells from the individual with the viral gene therapy agent at an MOI of about 1×10⁴ for about 24 hours, b) analyzing the innate immune cells for the expression of one or more cytokines wherein expression of a cytokine signature following incubation with the viral gene therapy agent indicates innate immunogenicity to the viral gene therapy agent. In some embodiments, the innate immune cell is a dendritic cell, a monocyte, a macrophage or a natural killer (NK) cell. In some embodiments, the innate immune cell is isolated from peripheral blood mononuclear cells from the individual. In some embodiments, the innate immune cell is a dendritic cell. In some embodiments, the dendritic cell is derived from a monocyte (e.g., a CD14+ monocyte) of the individual.

In some aspects, the invention provides methods for determining the innate immunogenicity to a viral gene therapy agent in an individual, the method comprising: a) incubating dendritic cells from the individual with the viral gene therapy agent at an MOI of about 1×10⁴ for about 24 hours, b) analyzing the dendritic cells for the expression of one or more cytokines wherein expression of a cytokine signature following incubation with the viral gene therapy agent indicates innate immunogenicity to the viral gene therapy agent. In some embodiments the dendritic cell is isolated from peripheral blood mononuclear cells from the individual. In some embodiments, the dendritic cell is derived from a monocyte (e.g., a CD14+ monocyte) of the individual. In some embodiments, the method further comprises isolating monocytes from the individual and incubating the monocytes in dendritic cell culture media to derive dendritic cells from the monocytes prior to incubating the dendritic cells with the gene therapy agent.

In some aspects, the invention provides methods for determining the innate immunogenicity of a viral gene therapy agent in an individual, the method comprising: a) obtaining peripheral blood mononuclear cells (PBMCs) from the individual, b) isolating innate immune cells from the PBMCs, c) incubating the innate immune cells with the viral gene therapy agent at an MOI of about 1×10⁴ for about 24 hours, c) analyzing the innate immune cells for the expression of one or more cytokines wherein expression of a cytokine signature following incubation with the viral gene therapy agent indicates innate immunogenicity to the viral gene therapy agent. In some embodiments, the innate immune cell is a dendritic cell, a monocyte, a macrophage or a natural killer (NK) cell. In some embodiments, the innate immune cell is a dendritic cell, wherein the dendritic cell is derived from a monocyte (e.g., a CD14+ monocyte) of the individual. In some embodiments, the method further comprises isolating monocytes from the individual and incubating the monocytes in dendritic cell culture media to derive dendritic cells from the monocytes prior to incubating the dendritic cells with the gene therapy agent.

In some aspects, the invention provides methods of determining the innate immunogenicity to a viral gene therapy agent in an individual, the method comprising a) incubating monocytes from the individual in dendritic cell culture media under conditions in which the monocytes differentiate into dendritic cells, b) incubating the dendritic cells with the viral gene therapy agent at an MOI of about 1×10³ to about 1×10⁵ for about 12 to about 36 hours, c) analyzing the dendritic cells for altered (e.g., increased, decreased) expression of one or more cytokines compared to a suitable control, whereby altered expression of the one or more cytokines produces a cytokine signature, wherein expression of the cytokine signature following incubation with the viral gene therapy agent indicates innate immunogenicity to the viral gene therapy agent in the individual, wherein the cytokine signature comprises increased expression of IL6, TNFα, and IL-1β. In some embodiments, the monocytes are obtained from peripheral mononuclear cells from the individual. In some embodiments, the monocytes are CD14+ monocytes. In some embodiments, the monocytes are incubated with the dendritic cell culture media for about 5 to about 10 days or about 7 to about 8 days to derive dendritic cells from the monocytes. In some embodiments, the monocytes are incubated with the dendritic cell culture media for about any of 3, 4, 5, 6, 7, 8, 9, 10, 11, 12, or more than 12 days to derive dendritic cells from the monocytes. In some embodiments, the dendritic cells are replated prior to the incubation with the gene therapy agent of step c). In some embodiments, the dendritic cells are replated into microwell dishes prior to incubation with the gene therapy agent. In some embodiments, the dendritic cells are incubated with the viral gene therapy agent at an MOI of any of about 1×10³, about 5×10³, about 1×10⁴, about 5×10⁴, or about 1×10⁵. In some embodiments, the dendritic cells are incubated with the viral gene therapy agent at an MOI of any of about 1×10³ to about 1×10⁵, about 5×10³ to about 1×10⁵, about 1×10⁴ to about 1×10⁵, about 5×10⁴ to about 1×10⁵, about 1×10³ to about 5×10⁴, about 5×10³ to about 5×10⁴, about 1×10⁴ to about 5×10⁴, about 1×10³ to about 1×10⁴, about 5×10³ to about 1×10⁴, or about 1×10³ to about 5×10³. In some embodiments, the dendritic cells are incubated with the viral gene therapy agent more than any of about 12 hours, about 18 hours, about 24 hours, about 30 hours, or about 36 hours. In some embodiments, the dendritic cells are incubated with the viral gene therapy agent for between any of about 12 hours and about 36 hours, about 18 hours and about 36 hours, about 24 hours and about 36 hours, about 30 hours and about 36 hours, about 12 hours and about 30 hours, about 18 hours and about 30 hours, about 24 hours and about 30 hours, about 12 hours and about 24 hours, about 18 hours and about 24 hours, or about 12 hours and about 18 hours.

In some aspects, the invention provides methods for determining the innate immunogenicity to a viral gene therapy agent in an individual, the method comprising: a) obtaining peripheral blood mononuclear cells (PBMCs) from the individual, b) isolating CD14+ monocytes from the PBMCs, c) incubating the monocytes in dendritic cell culture media for about 7-8 days to derive dendritic cells from the monocytes, d) replating the dendritic cells, e) incubating the dendritic cells with the viral gene therapy agent at an MOI of about 1×10⁴ for about 24 hours, f) analyzing the dendritic cells for the expression of one or more cytokines wherein expression of a cytokine signature following incubation with the viral gene therapy agent indicates innate immunogenicity to the viral gene therapy agent. In some embodiments, the cytokine signature comprises increased expression of IL6, TNFα, and IL-1β.

In some aspects, the invention provides methods of determining the innate immunogenicity to a non-viral gene therapy agent in an individual, the method comprising a) incubating monocytes from the individual in dendritic cell culture media under conditions in which the monocytes differentiate into dendritic cells, b) incubating the dendritic cells with the non-viral gene therapy agent at a concentration of about 1 ng/mL to about 1 mg/mL, c) analyzing the dendritic cells for altered (e.g., increased, decreased) expression of one or more cytokines compared to a suitable control, whereby altered expression of the one or more cytokines produces a cytokine signature, wherein expression of the cytokine signature following incubation with the non-viral gene therapy agent indicates innate immunogenicity to the non-viral gene therapy agent in the individual, wherein the cytokine signature comprises increased expression of IL6, TNFα, and IL-1β. In some embodiments, the monocytes are obtained from peripheral mononuclear cells from the individual. In some embodiments, the monocytes are CD14+ monocytes. In some embodiments, the monocytes are incubated with the dendritic cell culture media for about 5 to about 10 days or about 7 to about 8 days to derive dendritic cells from the monocytes. In some embodiments, the monocytes are incubated with the dendritic cell culture media for about any of 3, 4, 5, 6, 7, 8, 9, 10, 11, 12, or more than 12 days to derive dendritic cells from the monocytes. In some embodiments, the dendritic cells are replated prior to the incubation with the non-viral gene therapy agent of step c). In some embodiments, the dendritic cells are replated into microwell dishes prior to incubation with the gene therapy agent. In some embodiments, the innate immune cells are incubated with the non-viral gene therapy agent at a concentration of about 1 ng/mL to about 10 ng/mL, about 10 ng/mL to about 100 ng/mL, about 100 ng/mL to about 1 μg/mL, about 1 μg/mL to about 10 μg/mL, about 10 μg/mL to about 100 μg/mL, or about 100 μg/mL to about 1 mg/mL. In some embodiments, the dendritic cells are incubated with the non-viral gene therapy agent more than any of about 12 hours, about 18 hours, about 24 hours, about 30 hours, or about 36 hours. In some embodiments, the dendritic cells are incubated with the non-viral gene therapy agent for between any of about 12 hours and about 36 hours, about 18 hours and about 36 hours, about 24 hours and about 36 hours, about 30 hours and about 36 hours, about 12 hours and about 30 hours, about 18 hours and about 30 hours, about 24 hours and about 30 hours, about 12 hours and about 24 hours, about 18 hours and about 24 hours, or about 12 hours and about 18 hours.

In some aspects, the invention provides methods for determining the innate immunogenicity to a non-viral gene therapy agent in an individual, the method comprising: a) incubating innate immune cells from the individual with the non-viral gene therapy agent at a concentration of about 1 ng/mL to about 1 mg/mL for about 24 hours, b) analyzing the innate immune cells for the expression of one or more cytokines wherein expression of a cytokine signature following incubation with the non-viral gene therapy agent indicates innate immunogenicity to the non-viral gene therapy agent. In some embodiments, the innate immune cell is a dendritic cell, a monocyte, a macrophage or a natural killer (NK) cell. In some embodiments, the innate immune cell is isolated from peripheral blood mononuclear cells from the individual. In some embodiments, the innate immune cell is a dendritic cell. In some embodiments, the dendritic cell is derived from a monocyte (e.g., a CD14+ monocyte) of the individual.

In some aspects, the invention provides methods for determining the innate immunogenicity to a non-viral gene therapy agent in an individual, the method comprising: a) incubating dendritic cells from the individual with the non-viral gene therapy agent at a concentration of about 1 ng/mL to about 1 mg/mL for about 24 hours, b) analyzing the dendritic cells for the expression of one or more cytokines wherein expression of a cytokine signature following incubation with the non-viral gene therapy agent indicates innate immunogenicity to the non-viral gene therapy agent. In some embodiments, the dendritic cell is isolated from peripheral blood mononuclear cells from the individual. In some embodiments, the dendritic cell is derived from a monocyte (e.g., a CD14+ monocyte) of the individual. In some embodiments, the method further comprises isolating monocytes from the individual and incubating the monocytes in dendritic cell culture media to derive dendritic cells from the monocytes prior to incubating the dendritic cells with the gene therapy agent.

In some aspects, the invention provides methods for determining the innate immunogenicity to a non-viral gene therapy agent in an individual, the method comprising: a) obtaining peripheral blood mononuclear cells (PBMCs) from the individual, b) isolating innate immune cells from the PBMCs, c) incubating the innate immune cells with the non-viral gene therapy agent at a concentration of about 1 ng/mL to about 1 mg/mL for about 24 hours, c) analyzing the innate immune cells for the expression of one or more cytokines wherein expression of a cytokine signature following incubation with the non-viral gene therapy agent indicates innate immunogenicity to the non-viral gene therapy agent. In some embodiments, the innate immune cell is a dendritic cell, a monocyte, a macrophage or a natural killer (NK) cell. In some embodiments, the innate immune cell is a dendritic cell. In some embodiments, the dendritic cell is derived from a monocyte (e.g., a CD14+ monocyte) of the individual.

In some aspects, the invention provides methods for determining the innate immunogenicity to a non-viral gene therapy agent (e.g., an LNP) in an individual, the method comprising: a) obtaining peripheral blood mononuclear cells (PBMCs) from the individual, b) isolating CD14+ monocytes from the PBMCs, c) incubating the monocytes in dendritic cell culture media for about 7-8 days to derive dendritic cells from the monocytes, d) replating the dendritic cells, e) incubating the dendritic cells with the non-viral gene therapy agent at a concentration of about 1 ng/mL to about 1 mg/mL for about 24 hours, f) analyzing the dendritic cells for the expression of one or more cytokines wherein expression of a cytokine signature following incubation with the gene therapy agent indicates innate immunogenicity to the gene therapy agent. In some embodiments, the cytokine signature comprises increased expression of IL6, TNFα, and IL-1β.

In some embodiments, the monocytes are isolated from PBMCs from the individual. In some embodiments, the monocytes are CD14+ monocytes. In some embodiments, the monocytes are incubated with the dendritic cell culture media for about 5 to about 10 days or about 7 to about 8 days to derive dendritic cells from the monocytes. In some embodiments, the monocytes are incubated with the dendritic cell culture media for about any of 3, 4, 5, 6, 7, 8, 9, 10, 11, 12, or more than 12 days to derive dendritic cells from the monocytes. In some embodiments, the dendritic cells are replated prior to the incubation with the gene therapy agent of step c). In some embodiments, the dendritic cells are replated into microwell dishes prior to incubation with the gene therapy agent.

In some embodiment, the cells (e.g., PBMCs, monocytes, and/or dendritic cells) are isolated from a sample obtained from an individual or multiple individuals. As used herein a “sample” obtained from an individual includes any suitable sample that can be used to obtain PBMCs, monocytes, and/or dendritic cells such as blood, urine and/or tissue from one or more individuals. In some embodiments, the cells are obtained from blood of the individual (e.g., an individual in need of treatment with (or who has been treated with) the gene therapy agent). In some embodiments, the PBMCs are isolated from leukopaks. In some embodiments, PBMCs are isolated from blood using a Ficoll gradient. In some embodiments, the buffy coat containing white blood cells and platelets are collected from the Ficoll gradient. In some embodiments, the PBMCs are washed prior to culture. In some embodiments, the PBMCs are washed one, two, three, four, five or more than five times prior to culture. In some embodiments, the cells are washes with phosphate buffered saline prior to culture. In some embodiments, the PBS further contains Fetal Bovine Serum (FBS) or Fetal Calf Serum (FCS) prior to culture. In some embodiments, the FBC and/or FCS is added to the PBS at a final concentration between about 0.1% to about 10% (v/v). In some embodiments, the FBC and/or FCS is added to the PBS at a final concentration 1% (v/v).

In some embodiments of the invention, monocytes are isolated from PBMCs. In some embodiments, CD14+ monocytes are isolated from PBMCs. In some embodiments, monocytes (e.g., CD14+) monocytes are isolated from PBMCs by affinity purification. In some embodiments, CD14+ monocytes are isolated from PBMCs using an antibody that specifically binds CD14. In some embodiments, the anti-CD14 antibodies are affixed to a solid support such as a bead or a resin. In some embodiments, CD14+ antibodies are purified from PBMCs using an anti-CD14 antibody affixed to a magnetic bead. In some embodiments, the magnetic bead is a CD14 MicroBeads (Milteny Biotech).

In some embodiments of the invention, monocytes purified from PBMCs (e.g., CD14+ monocytes) are differentiated into dendritic cells. In some embodiments, monocytes from PBMCs are differentiated into dendritic cells by incubating the monocytes in the presence of cytokines that favor differentiation to dendritic cells. In some embodiments, dendritic cells are derived from monocytes by incubating the monocytes in ImmunoCult™ DC differentiation medium for about five days. In some embodiments, the ImmunoCult™ DC differentiation medium further comprises ImmunoCult™ DC differentiation supplement. In some embodiments, the ImmunoCult™ DC maturation supplement is added to the cultures after about five days. In some embodiments, differentiated dendritic cells are harvested on about day 7 for use in the assay of the invention.

In some embodiments, the innate immune cells (e.g., dendritic cells, monocytes, macrophages or NK cells) are incubated with the viral gene therapy agent at an MOI of about 1×10³ to about 1×10⁵ or about 1×10⁴. In some embodiments, the innate immune cells are incubated with the gene therapy agent at an MOI of less than about any of 1×10³, 5×10³, 1×10⁴, 5×10⁴, 1×10⁵, or 5×10⁵.

In some embodiments, the innate immune cells (e.g., dendritic cells, monocytes, macrophages or NK cells) are incubated with a non-viral gene therapy agent at a concentration of about 1 ng/mL to about 1 mg/mL. In some embodiments, the innate immune cells are incubated with the non-viral gene therapy agent at a concentration of about 1 ng/mL to about 10 ng/mL, about 10 ng/mL to about 100 ng/mL, about 100 ng/mL to about 1 μg/mL, about 1 μg/mL to about 10 μg/mL, about 10 μg/mL to about 100 μg/mL, or about 100 μg/mL to about 1 mg/mL.

In some embodiments, the innate immune cells (e.g., dendritic cells, monocytes, macrophages or NK cells)\ are incubated with the gene therapy agent for about 12 hours to about 36 hours or about 24 hours. In some embodiments, the dendritic cells are incubated with the gene therapy agent for between about 6 hours and about 48 hours, about 6 hours and about 36 hours, about 6 hours and about 24 hours, about 6 hours and about 18 hours, about 6 hours and about 12 hours, about 12 hours and about 48 hours, about 12 hours and about 36 hours, about 12 hours and about 24 hours, about 12 hours and about 18 hours, about 18 hours and about 48 hours, about 18 hours and about 36 hours, about 18 hours and about 24 hours, about 24 hours and about 48 hours, about 24 hours and about 36 hours, or about 36 hours and about 48 hours.

In some embodiments, a cytokine signature is determined for a gene therapy agent in a particular immune cell (e.g., a dendritic cell, a monocyte, a macrophage, an NK cell, etc.) by contacting the particular immune cells from one individual or a plurality of individuals with a gene therapy agent and determining altered expression (e.g., increased/decreased) of one or more cytokines associated with an innate immune response, wherein a commonality in changes in expression (e.g., increased or decreased expression) in the one or more cytokines indicates the presence of a cytokine signature. In some embodiments, the cytokines associated with an innate immune response are associated with a toll-like receptor (TLR) pathway (e.g., a TLR2, TLR3, TLR4 or TLR9 pathway). In some embodiments, the cytokine signature comprises changes in expression in more than any of 1, 2, 3, 4, 5, 6, 7, 8, 9, or 10 cytokines. In some embodiments, the plurality of individuals comprises more than any of 1, 2, 3, 4, 5, 6, 7, 8, 9, or 10 individuals. In some embodiments, the commonality of changes in expression comprises similar changes in expression levels of cytokines in innate immune cells in greater than about 25%, 50%, 75% or 90% of the individuals in the plurality of individuals.

In some embodiments, the invention provides methods of determining the cytokine signature of a gene therapy agent comprising a) incubating one or more innate immune cell (e.g., a dendritic cell, a monocyte, a macrophage, an NK cell, etc.) from one or more individuals with the gene therapy agent, b) analyzing the one or more innate immune cells for altered (e.g., increased/decreased) expression of one or more cytokines compared to a suitable control, wherein the altered expression of one or more cytokines in step b) indicates the cytokine signature of the gene therapy agent. In some embodiments, the innate immune cell is obtained from a blood sample from the one or more individuals. In some embodiments, the innate immune cell is obtained from a PBMCs from the one or more individuals. In some embodiments, the gene therapy agent is a viral particle or a lipid nanoparticle. In some embodiments, the gene therapy agent is an adeno-associated virus (AAV) particle, an adenovirus particle, a lentivirus particle, or a herpes simplex virus (HAV) particle. In some embodiments, the gene therapy agent is a lipid nanoparticle or a liposome.

In some embodiments, the cytokine signature comprises increased expression of one or more of IL6, TNFα, IL-1β, MCP1 and MIP-1α. In some embodiments, the cytokine signature comprises increased expression of two or more of IL6, TNFα, IL-1β, MCP1 and MIP-1α. In some embodiments, the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β.

In some embodiments, the cytokine signature comprises increased expression of one or more of IP10, MIP1b, CXCL9, and IL2. In some embodiments, the cytokine signature comprises increased expression of two or more of IP10, MIP1b, CXCL9, and IL2. In some embodiments, the cytokine signature comprises increased expression of three or more of IP10, MIP1b, CXCL9, and IL2. In some embodiments, the cytokine signature comprises increased expression of IP10, MIP1b, CXCL9, and IL2. In some embodiments, the cytokine signature comprises increased expression of IP10, MIP1b, CXCL9, IL2, and IL6. In some embodiments, the cytokine signature comprises increased expression of IP10, MIP1b, CXCL9, IL2, and TNFα. In some embodiments, the cytokine signature comprises increased expression of IP10, MIP1b, CXCL9, IL2, IL6, and TNFα.

In some embodiments, the innate immune cell is a dendritic cell and the cytokine signature comprises increased expression of one or more of IL6, TNFα, IL-1β, MCP1 and MIP-1α. In some embodiments, the cytokine signature comprises increased expression of two or more of IL6, TNFα, IL-1β, MCP1 and MIP-1α. In some embodiments, the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β.

In some embodiments, the innate immune cell is a dendritic cell and the cytokine signature comprises increased expression of one or more of IP10, MIP1b, CXCL9, and IL2. In some embodiments, the cytokine signature comprises increased expression of two or more of IP10, MIP1b, CXCL9, and IL2. In some embodiments, the cytokine signature comprises increased expression of three or more of IP10, MIP1b, CXCL9, and IL2. In some embodiments, the cytokine signature comprises increased expression of IP10, MIP1b, CXCL9, and IL2. In some embodiments, the cytokine signature comprises increased expression of IP10, MIP1b, CXCL9, IL2, and IL6. In some embodiments, the cytokine signature comprises increased expression of IP10, MIP1b, CXCL9, IL2, and TNFα. In some embodiments, the cytokine signature comprises increased expression of IP10, MIP1b, CXCL9, IL2, IL6, and TNFα.

In some embodiments, expression of the cytokines in the cytokine signature is increased compared to expression of the cytokines in the cytokine signature from innate immune cells incubated in the absence of the gene therapy agent or compared to expression of the cytokines in the cytokine signature from innate immune cells prior to incubation with the gene therapy agent, wherein the cytokine signature comprises increased expression of one or more of IL6, TNFα, IL-1β, MCP1 and MIP-1α. In some embodiments, the cytokine signature comprises increased expression of two or more of IL6, TNFα, IL-1β, MCP1 and MIP-1α. In some embodiments, the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β. In some embodiments, an increase in expression of any one of about 10%, about 20%, about 25%, about 50%, about 75%, about 100%, or more than 100% identifies an individual for treatment with a gene therapy agent and an innate immune response modulator (e.g., an IRAK modulator).

In some embodiments, expression of the cytokines in the cytokine signature is increased compared to expression of the cytokines in the cytokine signature from innate immune cells incubated in the absence of the gene therapy agent or compared to expression of the cytokines in the cytokine signature from innate immune cells prior to incubation with the gene therapy agent, wherein the cytokine signature comprises increased expression of one or more of IP10, MIP1b, CXCL9, and IL2. In some embodiments, the cytokine signature comprises increased expression of two or more of IP10, MIP1b, CXCL9, and IL2. In some embodiments, the cytokine signature comprises increased expression of three or more of IP10, MIP1b, CXCL9, and IL2. In some embodiments, the cytokine signature comprises increased expression of IP10, MIP1b, CXCL9, and IL2. In some embodiments, the cytokine signature comprises increased expression of IP10, MIP1b, CXCL9, IL2, and IL6. In some embodiments, the cytokine signature comprises increased expression of IP10, MIP1b, CXCL9, IL2, and TNFα. In some embodiments, the cytokine signature comprises increased expression of IP10, MIP1b, CXCL9, IL2, IL6, and TNFα. In some embodiments, an increase in expression of any one of about 10%, about 20%, about 25%, about 50%, about 75%, about 100%, or more than 100% identifies an individual for treatment with a gene therapy agent and an innate immune response modulator (e.g., an IRAK modulator).

In some embodiments, expression of the cytokines in the cytokine signature is increased compared to expression of the cytokines in the cytokine signature from dendritic cells incubated in the absence of the gene therapy agent or compared to expression of the cytokines in the cytokine signature from innate immune cells prior to incubation with the gene therapy agent, wherein the cytokine signature comprises increased expression of one or more of IL6, TNFα, IL-1β, MCP1 and MIP-1α. In some embodiments, the cytokine signature comprises increased expression of two or more of IL6, TNFα, IL-1β, MCP1 and MIP-1α. In some embodiments, the cytokine signature comprises increased expression of three or more of IL6, TNFα, IL-1β, MCP1 and MIP-1α. In some embodiments, the cytokine signature comprises increased expression of four or more of IL6, TNFα, IL-1β, MCP1 and MIP-1α. In some embodiments, the cytokine signature comprises increased expression of IL6, TNFα, IL-1β, MCP1 and MIP-1α. In some embodiments, the cytokine signature comprises increased expression of IL6, TNFα, and IL-1β. In some embodiments, an increase in expression of any one of about 10%, about 20%, about 25%, about 50%, about 75%, about 100%, or more than 100% identifies an individual for treatment with a gene therapy agent and an innate immune response modulator (e.g., an IRAK modulator).

In some embodiments, expression of the cytokines in the cytokine signature is increased compared to expression of the cytokines in the cytokine signature from dendritic cells incubated in the absence of the gene therapy agent or compared to expression of the cytokines in the cytokine signature from innate immune cells prior to incubation with the gene therapy agent, wherein the cytokine signature comprises increased expression of one or more of IP10, MIP1b, CXCL9, and IL2. In some embodiments, the cytokine signature comprises increased expression of two or more of IP10, MIP1b, CXCL9, and IL2. In some embodiments, the cytokine signature comprises increased expression of three or more of IP10, MIP1b, CXCL9, and IL2. In some embodiments, the cytokine signature comprises increased expression of IP10, MIP1b, CXCL9, and IL2. In some embodiments, the cytokine signature comprises increased expression of IP10, MIP1b, CXCL9, IL2, and IL6. In some embodiments, the cytokine signature comprises increased expression of IP10, MIP1b, CXCL9, IL2, and TNFα. In some embodiments, the cytokine signature comprises increased expression of IP10, MIP1b, CXCL9, IL2, IL6, and TNFα. In some embodiments, an increase in expression of any one of about 10%, about 20%, about 25%, about 50%, about 75%, about 100%, or more than 100% identifies an individual for treatment with a gene therapy agent and an innate immune response modulator (e.g., an IRAK modulator).

### Gene Therapy Agents

In some aspects, the invention provides methods for determining innate immunogenicity to a gene therapy agent in an individual improved gene therapy; for example, by identifying an individual in which an agent that modulates an innate immune response to a gene therapy agent is administered in combination with (sequentially (before or after), simultaneously) the gene therapy agent. As used herein “modulating” an innate immune response refers to e.g., partially or completely stimulating, delaying suppressing and/or inhibiting an innate immune response. In some embodiments, an IRAK modulator is used to inhibit an innate immune response in the individual. In some embodiments, the gene therapy agent is a viral particle or a lipid nanoparticle. In some embodiments, the gene therapy agent is an adeno-associated virus (AAV) particle, an adenovirus particle, a lentivirus particle, or a herpes simplex virus (HAV) particle. In some embodiments, the gene therapy agent is a lipid nanoparticle or a liposome.

### AAV

In some embodiments, the invention provides methods for determining innate immunogenicity to an AAV particle in an individual. In an AAV particle for gene therapy, a recombinant AAV (rAAV) genome encoding a heterologous nucleic acid (e.g., a therapeutic transgene) is encapsidated in an AAV capsid. In some embodiments, the viral genome comprises a heterologous nucleic acid and/or one or more of the following components, operatively linked in the direction of transcription, control sequences including transcription initiation and termination sequences, thereby forming an expression cassette.

In some embodiments, the rAAV genome comprises one or more AAV inverted terminal repeat (ITR) sequences (typically two AAV ITR sequences). For example, an expression cassette may be flanked on the 5′ and 3′ end by at least one functional AAV ITR sequence. By “functional AAV ITR sequences”, it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant viral genomes comprise at least all of the sequences of AAV essential for encapsidation into the AAV capsid and the physical structures for infection by the AAV particle. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810. Use of any AAV serotype is considered within the scope of the present invention. In some embodiments, a rAAV vector is a vector derived from an AAV serotype, including without limitation, AAV1, AAV2, AAV3, AAV4, AAV5, AAV6, AAV7, AAV8, AAVrh8, AAVrh8R, AAV9, AAV10, AAVrh10, AAV11, AAV12, AAV LK03, AAV2R471A, AAV DJ, AAV DJ8, a goat AAV, bovine AAV, or mouse AAV ITRs or the like. In some embodiments, the nucleic acid in the AAV (e.g., an rAAV vector) comprises an ITR of AAV1, AAV2, AAV3, AAV4, AAV5, AAV6, AAV7, AAV8, AAVrh8, AAVrh8R, AAV9, AAV10, AAVrh10, AAV11, AAV12, AAV LK03, AAV2R471A, AAV DJ, AAV DJ8, a goat AAV, bovine AAV, or mouse AAV ITRs or the like. In some embodiments, the AAV particle comprises an AAV vector encoding a heterologous transgene flanked by one or more AAV ITRs.

In some embodiments, the AAV particle comprises a capsid protein selected from an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LK03 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof. By “functional variant” of an AAV capsid, it is meant that the variant capsid is capable of packaging an AAV genome to generate an infectious AAV virion. In further embodiments, a rAAV particle comprises capsid proteins of an AAV serotype from Clades A-F.

In some aspects, the invention provides AAV particles comprising a recombinant self-complementing genome (e.g., a self-complementary or self-complimenting AAV vector). AAV viral particles with self-complementing vector genomes and methods of use of self-complementing rAAV genomes are described in U.S. Pat. Nos. 6,596,535; 7,125,717; 7,465,583; 7,785,888; 7,790,154; 7,846,729; 8,093,054; and 8,361,457; and Wang Z., et al., (2003) Gene Ther 10:2105-2111, each of which are incorporated herein by reference in its entirety. An AAV particle comprising a self-complementing genome will quickly form a double stranded DNA molecule by virtue of its partially complementing sequences (e.g., complementing coding and non-coding strands of a heterologous nucleic acid). In some embodiments, the vector comprises a first nucleic acid sequence encoding a heterologous nucleic acid and a second nucleic acid sequence encoding a complement of the nucleic acid, where the first nucleic acid sequence can form intrastrand base pairs with the second nucleic acid sequence along most or all of its length.

In some embodiments, the first heterologous nucleic acid sequence and a second heterologous nucleic acid sequence are linked by a mutated ITR (e.g., the right ITR). In some embodiments, the ITR comprises the polynucleotide sequence 5′-CACTCCCTCTCTGCGCGCTCGCTCGCTCACTGAGGCC GGGCGACCAAAGGTCGCCCACGCCCGGGCTTTGCCCGGGCG-3′ (SEQ ID NO:). The mutated ITR comprises a deletion of the D region comprising the terminal resolution sequence. As a result, on replicating a rAAV genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

Different AAV serotypes are used to optimize transduction of particular target cells or to target specific cell types within a particular target tissue (e.g., a diseased tissue). An AAV particle can comprise viral proteins and viral nucleic acids of the same serotype or a mixed serotype. For example, an AAV particle may contain one or more ITRs and capsid derived from the same AAV serotype, or an AAV particle may contain one or more ITRs derived from a different AAV serotype than capsid of the AAV particle.

In some embodiments, the AAV capsid comprises a mutation, e.g., the capsid comprises a mutant capsid protein. In some embodiments, the mutation is a tyrosine mutation or a heparin binding mutation. In some embodiments, a mutant capsid protein maintains the ability to form an AAV capsid. In some embodiments, the AAV particle comprises an AAV2 or AAV5 tyrosine mutant capsid (see, e.g., Zhong L. et al., (2008) Proc Natl Acad Sci USA 105(22):7827-7832), such as a mutation in Y444 or Y730 (numbering according to AAV2). In further embodiments, the AAV particle comprises capsid proteins of an AAV serotype from Clades A-F (Gao, et al., J. Virol. 2004, 78(12):6381).

Numerous methods are known in the art for production of AAV particles for gene therapy, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids (Urabe, M. et al., (2002) Human Gene Therapy 13(16):1935-1943; Kotin, R. (2011) Hum Mol Genet. 20(R1): R2-R6). AAV production cultures for the production of AAV particles all require; 1) suitable host cells, 2) suitable helper virus function, 3) AAV rep and cap genes and gene products; 4) a nucleic acid (such as a therapeutic nucleic acid) flanked by at least one AAV ITR sequences; and 5) suitable media and media components to support AAV production. In some embodiments, the suitable host cell is a primate host cell. In some embodiments, the suitable host cell is a human-derived cell lines such as HeLa, A549, 293, or Perc.6 cells. In some embodiments, the suitable helper virus function is provided by wild-type or mutant adenovirus (such as temperature sensitive adenovirus), herpes virus (HSV), baculovirus, or a plasmid construct providing helper functions. In some embodiments, the AAV rep and cap gene products may be from any AAV serotype. In general, but not obligatory, the AAV rep gene product is of the same serotype as the ITRs of the rAAV genome as long as the rep gene products may function to replicated and package the rAAV genome. Suitable media known in the art may be used for the production of AAV particles. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

One method for producing AAV particles is the triple transfection method. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified. As such, in some embodiments, the AAV particle was produced by triple transfection of a nucleic acid encoding the AAV vector, a nucleic acid encoding AAV rep and cap, and a nucleic acid encoding AAV helper virus functions into a host cell, wherein the transfection of the nucleic acids to the host cells generates a host cell capable of producing AAV particles.

In some embodiments, AAV particles may be produced by a producer cell line method (see Martin et al., (2013) Human Gene Therapy Methods 24:253-269; U.S. PG Pub. No. US2004/0224411; and Liu, X. L. et al. (1999) Gene Ther. 6:293-299). Briefly, a cell line (e.g., a HeLa, 293, A549, or Perc.6 cell line) may be stably transfected with a plasmid containing a rep gene, a capsid gene, and a vector genome comprising a promoter-heterologous nucleic acid sequence. Cell lines may be screened to select a lead clone for AAV production, which may then be expanded to a production bioreactor and infected with a helper virus (e.g., an adenovirus or HSV) to initiate AAV production. Virus may subsequently be harvested, adenovirus may be inactivated (e.g., by heat) and/or removed, and the AAV particles may be purified. As such, in some embodiments, the AAV particle was produced by a producer cell line comprising one or more of nucleic acid encoding the rAAV genome, a nucleic acid encoding AAV rep and cap, and a nucleic acid encoding AAV helper virus functions.

In some embodiments, the nucleic acid encoding AAV rep and cap genes and/or the AAV viral genome are stably maintained in the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and AAV genome are introduced into a cell on the same plasmid. In other embodiments, the AAV rep, AAV cap, and rAAV genome are introduced into a cell on different plasmids. In some embodiments, a cell line stably transfected with a plasmid maintains the plasmid for multiple passages of the cell line (e.g., 5, 10, 20, 30, 40, 50 or more than 50 passages of the cell). For example, the plasmid(s) may replicate as the cell replicates, or the plasmid(s) may integrate into the cell genome. A variety of sequences that enable a plasmid to replicate autonomously in a cell (e.g., a human cell) have been identified (see, e.g., Krysan, P. J. et al. (1989) Mol. Cell Biol. 9:1026-1033). In some embodiments, the plasmid(s) may contain a selectable marker (e.g., an antibiotic resistance marker) that allows for selection of cells maintaining the plasmid. Selectable markers commonly used in mammalian cells include without limitation blasticidin, G418, hygromycin B, zeocin, puromycin, and derivatives thereof. Methods for introducing nucleic acids into a cell are known in the art and include without limitation viral transduction, cationic transfection (e.g., using a cationic polymer such as DEAE-dextran or a cationic lipid such as lipofectamine), calcium phosphate transfection, microinjection, particle bombardment, electroporation, and nanoparticle transfection (for more details, see e.g., Kim, T. K. and Eberwine, J. H. (2010) Anal. Bioanal. Chem. 397:3173-3178).

In some embodiments, the producer cell line is derived from a primate cell line (e.g., a non-human primate cell line, such as a Vero or FRhL-2 cell line). In some embodiments, the cell line is derived from a human cell line. In some embodiments, the producer cell line is derived from HeLa, 293, A549, or PERC.6® (Crucell) cells. For example, prior to introduction and/or stable maintenance/integration of nucleic acid encoding AAV rep and cap genes and/or the rAAV genome into a cell line to generate a producer cell line, the cell line is a HeLa, 293, A549, or PERC.6® (Crucell) cell line, or a derivative thereof.

In some embodiments, the producer cell line is adapted for growth in suspension. As is known in the art, anchorage-dependent cells are typically not able to grow in suspension without a substrate, such as microcarrier beads. Adapting a cell line to grow in suspension may include, for example, growing the cell line in a spinner culture with a stirring paddle, using a culture medium that lacks calcium and magnesium ions to prevent clumping (and optionally an antifoaming agent), using a culture vessel coated with a siliconizing compound, and selecting cells in the culture (rather than in large clumps or on the sides of the vessel) at each passage.

AAV particles of the invention may be harvested from AAV production cultures by lysis of the host cells of the production culture or by harvest of the spent media from the production culture, provided the cells are cultured under conditions known in the art to cause release of AAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the AAV particles are purified. The term “purified” as used herein includes a preparation of AAV particles devoid of at least some of the other components that may also be present where the AAV particles naturally occur or are initially prepared from. Thus, for example, isolated AAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the AAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, and a 0.2 μm Filter Opticap XL1O Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art.

In some embodiments, the AAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

AAV particles may be isolated or purified using one or more of the following purification steps: equilibrium centrifugation; flow-through anionic exchange filtration; tangential flow filtration (TFF) for concentrating the AAV particles; AAV capture by apatite chromatography; heat inactivation of helper virus; AAV capture by hydrophobic interaction chromatography; buffer exchange by size exclusion chromatography (SEC); nanofiltration; and AAV capture by anionic exchange chromatography, cationic exchange chromatography, or affinity chromatography. These steps may be used alone, in various combinations, or in different orders. In some embodiments, the method comprises all the steps in the order as described below. Methods to purify AAV particles are found, for example, in Xiao et al., (1998) Journal of Virology 72:2224-2232; U.S. Pat. Nos. 6,989,264 and 8,137,948; and WO 2010/148143.

### Adenovirus

In some embodiments, the invention provides methods for determining innate immunity to an adenovirus particle in an individual. Adenoviral vectors for gene therapy are typically adenoviral particles with a recombinant adenovirus (rAd) genome comprising one or more heterologous sequences (i.e., nucleic acid sequence not of adenoviral origin) between two adenoviral ITRs encapsidated into an adenoviral capsid. In some embodiments, the heterologous sequence encodes a therapeutic transgene. In some embodiments, the rAd genome lacks or contains a defective copy of one or more E1 genes, which renders the adenovirus replication-defective. Adenoviruses include a linear, double-stranded DNA genome within a large (˜950 Å), non-enveloped icosahedral capsid. Adenoviruses have a large genome that can incorporate more than 30 kb of heterologous sequence (e.g., in place of the E1 and/or E3 region), making them uniquely suited for use with larger heterologous genes. They are also known to infect dividing and non-dividing cells and do not naturally integrate into the host genome (although hybrid variants may possess this ability). In some embodiments, the adenoviral vector may be a first generation adenoviral vector with a heterologous sequence in place of E1. In some embodiments, the adenoviral vector may be a second generation adenoviral vector with additional mutations or deletions in E2A, E2B, and/or E4. In some embodiments, the adenoviral vector may be a third generation or gutted adenoviral vector that lacks all viral coding genes, retaining only the ITRs and packaging signal and requiring a helper adenovirus in trans for replication, and packaging. Adenoviral particles have been investigated for use as vectors for transient transfection of mammalian cells as well as gene therapy vectors. For further description, see, e.g., Danthinne, X. and Imperiale, M. J. (2000) Gene Ther. 7:1707-14 and Tatsis, N. and Ertl, H. C. (2004) Mol. Ther. 10:616-29.

In some embodiments, the adenoviral particle comprises a rAd genome comprising a therapeutic transgene. Use of any adenovirus serotype is considered within the scope of the present invention. In some embodiments, the adenoviral particle is derived from an adenovirus serotype, including without limitation, AdHu2, AdHu 3, AdHu4, AdHu5, AdHu7, AdHu11, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, and porcine Ad type 3. The adenoviral particle also comprises capsid proteins. In some embodiments, the adenoviral particle includes one or more foreign viral capsid proteins. Such combinations may be referred to as pseudotyped adenoviral particles. In some embodiments, foreign viral capsid proteins used in pseudotyped adenoviral particles are derived from a foreign virus or from another adenovirus serotype. In some embodiments, the foreign viral capsid proteins are derived from, including without limitation, reovirus type 3. Examples of vector and capsid protein combinations used in pseudotyped adenovirus particles can be found in the following references (Tatsis, N. et al. (2004) Mol. Ther. 10(4):616-629 and Ahi, Y. et al. (2011) Curr. Gene Ther. 11(4):307-320). Different adenovirus serotypes can be used to optimize transduction of particular target cells or to target specific cell types within a particular target tissue (e.g., a diseased tissue). Tissues or cells targeted by specific adenovirus serotypes, include without limitation, lung (e.g. HuAd3), spleen and liver (e.g. HuAd37), smooth muscle, synoviocytes, dendritic cells, cardiovascular cells, tumor cell lines (e.g. HuAd11), and dendritic cells (e.g. HuAd5 pseudotyped with reovirus type 3, HuAd30, or HuAd35). For further description, see Ahi, Y. et al. (2011) Curr. Gene Ther. 11(4):307-320, Kay, M. et al. (2001) Nat. Med. 7(1):33-40, and Tatsis, N. et al. (2004) Mol. Ther. 10(4):616-629.

Numerous methods are known in the art for production of adenoviral particles. For example, for a gutted adenoviral vector, the adenoviral vector genome and a helper adenovirus genome may be transfected into a packaging cell line (e.g., a 293 cell line). In some embodiments, the helper adenovirus genome may contain recombination sites flanking its packaging signal, and both genomes may be transfected into a packaging cell line that expresses a recombinase (e.g., the Cre/loxP system may be used), such that the adenoviral vector of interest is packaged more efficiently than the helper adenovirus (see, e.g., Alba, R. et al. (2005) Gene Ther. 12 Suppl 1:S18-27). Adenoviral vectors may be harvested and purified using standard methods, such as those described herein.

### Lentivirus

In some embodiments, the invention provides methods for determining innate immunogenicity to a lentivirus particle in an individual. Lentiviral vectors for gene therapy are typically lentiviral particles with a recombinant lentivirus genome comprising one or more heterologous sequences (i.e., nucleic acid sequence not of lentiviral origin) between two long terminal repeats (LTRs). In some embodiments, the heterologous sequence encodes a therapeutic transgene. Lentiviruses are positive-sense, ssRNA retroviruses with a genome of approximately 10 kb. Lentiviruses integrate into the genome of dividing and non-dividing cells. Lentiviral particles may be produced, for example, by transfecting multiple plasmids (typically the lentiviral genome and the genes required for replication and/or packaging are separated to prevent viral replication) into a packaging cell line, which packages the modified lentiviral genome into lentiviral particles. In some embodiments, a lentiviral particle may refer to a first generation vector that lacks the envelope protein. In some embodiments, a lentiviral particle may refer to a second-generation vector that lacks all genes except the gag/pol and tat/rev regions. In some embodiments, a lentiviral particle may refer to a third generation vector that only contains the endogenous rev, gag, and pol genes and has a chimeric LTR for transduction without the tat gene (see Dull, T. et al. (1998) J. Virol. 72:8463-71). For further description, see Durand, S. and Cimarelli, A. (2011) Viruses 3:132-59.

Use of any lentiviral vector is considered within the scope of the present invention. In some embodiments, the lentiviral vector is derived from a lentivirus including, without limitation, human immunodeficiency virus-1 (HIV-1), human immunodeficiency virus-2 (HIV-2), simian immunodeficiency virus (SIV), feline immunodeficiency virus (FIV), equine infectious anemia virus (EIAV), bovine immunodeficiency virus (BIV), Jembrana disease virus (JDV), visna virus (VV), and caprine arthritis encephalitis virus (CAEV). The lentiviral particle also comprises capsid proteins. In some embodiments, the lentivirus particles include one or more foreign viral capsid proteins. Such combinations may be referred to as pseudotyped lentiviral particles. In some embodiments, foreign viral capsid proteins used in pseudotyped lentiviral particles are derived from a foreign virus. In some embodiments, the foreign viral capsid protein used in pseudotyped lentiviral particles is Vesicular stomatitis virus glycoprotein (VSV-GP). VSV-GP interacts with a ubiquitous cell receptor, providing broad tissue tropism to pseudotyped lentiviral particles. In addition, VSV-GP is thought to provide higher stability to pseudotyped lentiviral particles. In other embodiments, the foreign viral capsid proteins are derived from, including without limitation, Chandipura virus, Rabies virus, Mokola virus, Lymphocytic choriomeningitis virus (LCMV), Ross River virus (RRV), Sindbis virus, Semliki Forest virus (SFV), Venezuelan equine encephalitis virus, Ebola virus Reston, Ebola virus Zaire, Marburg virus, Lassa virus, Avian leukosis virus (ALV), Jaagsiekte sheep retrovirus (JSRV), Moloney Murine leukemia virus (MLV), Gibbon ape leukemia virus (GALV), Feline endogenous retrovirus (RD114), Human T-lymphotropic virus 1 (HTLV-1), Human foamy virus, Maedi-visna virus (MVV), SARS-CoV, Sendai virus, Respiratory syncytia virus (RSV), Human parainfluenza virus type 3, Hepatitis C virus (HCV), Influenza virus, Fowl plague virus (FPV), or Autographa californica multiple nucleopolyhedro virus (AcMNPV). Examples of vector and capsid protein combinations used in pseudotyped lentivirus particles can be found, for example, in Cronin, J. et al. (2005). Curr. Gene Ther. 5(4):387-398. Different pseudotyped lentiviral particles can be used to optimize transduction of particular target cells or to target specific cell types within a particular target tissue (e.g., a diseased tissue). For example, tissues targeted by specific pseudotyped lentiviral particles, include without limitation, liver (e.g. pseudotyped with a VSV-G, LCMV, RRV, or SeV F protein), lung (e.g. pseudotyped with an Ebola, Marburg, SeV F and HN, or JSRV protein), pancreatic islet cells (e.g. pseudotyped with an LCMV protein), central nervous system (e.g. pseudotyped with a VSV-G, LCMV, Rabies, or Mokola protein), retina (e.g. pseudotyped with a VSV-G or Mokola protein), monocytes or muscle (e.g. pseudotyped with a Mokola or Ebola protein), hematopoietic system (e.g. pseudotyped with an RD114 or GALV protein), or cancer cells (e.g. pseudotyped with a GALV or LCMV protein). For further description, see Cronin, J. et al. (2005). Curr. Gene Ther. 5(4):387-398 and Kay, M. et al. (2001) Nat. Med. 7(1):33-40.

Numerous methods are known in the art for production of lentiviral particles. For example, for a third-generation lentiviral vector, a vector containing the recombinant lentiviral genome of interest with gag and pol genes may be co-transfected into a packaging cell line (e.g., a 293 cell line) along with a vector containing a rev gene. The recombinant lentiviral genome of interest also contains a chimeric LTR that promotes transcription in the absence of Tat (see Dull, T. et al. (1998) J. Virol. 72:8463-71). Lentiviral vectors may be harvested and purified using methods (e.g., Segura M M, et al., (2013) Expert Opin Biol Ther. 13(7):987-1011) described herein.

### HSV

In some embodiments, the invention provides methods for determining innate immunogenicity to a HSV particle in an individual. HSV vectors for gene therapy are typically HSV particles with a recombinant HSV genome comprising one or more heterologous sequences (i.e., nucleic acid sequence not of HSV origin) between two terminal repeats (TRs). In some embodiments, the heterologous sequence encodes a therapeutic transgene. HSV is an enveloped, double-stranded DNA virus with a genome of approximately 152 kb. Advantageously, approximately half of its genes are nonessential and may be deleted to accommodate heterologous sequence. HSV particles infect non-dividing cells. In addition, they naturally establish latency in neurons, travel by retrograde transport, and can be transferred across synapses, making them advantageous for transfection of neurons and/or gene therapy approaches involving the nervous system. In some embodiments, the HSV particle may be replication-defective or replication-competent (e.g., competent for a single replication cycle through inactivation of one or more late genes). For further description, see Manservigi, R. et al. (2010) Open Virol. J. 4:123-56.

In some embodiments, the HSV particle comprising a recombinant HSV genome comprising a transgene. Use of any HSV vector is considered within the scope of the present invention. In some embodiments, the HSV vector is derived from a HSV serotype, including without limitation, HSV-1 and HSV-2. The HSV particle also comprises capsid proteins. In some embodiments, the HSV particles include one or more foreign viral capsid proteins. Such combinations may be referred to as pseudotyped HSV particles. In some embodiments, foreign viral capsid proteins used in pseudotyped HSV particles are derived from a foreign virus or from another HSV serotype. In some embodiments, the foreign viral capsid protein used in a pseudotyped HSV particle is a Vesicular stomatitis virus glycoprotein (VSV-GP). VSV-GP interacts with a ubiquitous cell receptor, providing broad tissue tropism to pseudotyped HSV particles. In addition, VSV-GP is thought to provide higher stability to pseudotyped HSV particles. In other embodiments, the foreign viral capsid protein may be from a different HSV serotype. For example, an HSV-1 vector may contain one or more HSV-2 capsid proteins. Different HSV serotypes can be used to optimize transduction of particular target cells or to target specific cell types within a particular target tissue (e.g., a diseased tissue). Tissues or cells targeted by specific adenovirus serotypes include without limitation, central nervous system and neurons (e.g. HSV-1). For further description, see Manservigi, R. et al. (2010) Open Virol J 4:123-156, Kay, M. et al. (2001) Nat. Med. 7(1):33-40, and Meignier, B. et al. (1987) J. Infect. Dis. 155(5):921-930.

Numerous methods are known in the art for production of HSV particles. HSV vectors may be harvested and purified using standard methods, such as those described herein. For example, for a replication-defective HSV vector, an HSV genome of interest that lacks all of the immediate early (IE) genes may be transfected into a complementing cell line that provides genes required for virus production, such as ICP4, ICP27, and ICP0 (see, e.g., Samaniego, L. A. et al. (1998) J. Virol. 72:3307-20). HSV vectors may be harvested and purified using methods described (e.g., Goins, W F et al., (2014) Herpes Simplex Virus Methods in Molecular Biology 1144:63-79).

### Nonviral Gene Therapy Agents

In some embodiments, the invention provides methods for determining an innate immune response to a non-viral gene therapy agent. Non-viral vector delivery systems include DNA plasmids, naked nucleic acid, and nucleic acid complexed to a delivery system. For example, the vector may be complexed to a lipid (e.g., a cationic or neutral lipid), a liposome, a polycation, a lipid nanoparticle, or an agent that enhances the cellular uptake of nucleic acid. The nucleic acid may be complexed to an agent suitable for any of the delivery methods described herein. In some embodiments, the nucleic acid encodes a therapeutic transgene.

Lipid nanoparticles for gene therapy typically comprise a vector genome encapsulated in a lipid particle or a vector genome complexed with a lipid. In some embodiments, the heterologous sequence encodes a therapeutic transgene. In some embodiments, the vector genome is formulated in a lipoplex nanoparticle or liposome. In some embodiments, a lipoplex nanoparticle formulation for the gene therapy agent comprises the synthetic cationic lipid (R)—N,N,N-trimethyl-2,3-dioleyloxy-1-propanaminium chloride (DOTMA) and the phospholipid 1,2-dioleoyl-sn-glycero-3-phosphoethanolamine (DOPE). In some embodiments, the DOTMA/DOPE liposomal component is optimized for delivery and targeting of cells in the individual.

In some embodiments, nucleic acid comprising the vector genome is mixed with a pharmaceutical composition comprising one or more cationic lipids, including, e.g., (R)—N,N,N-trimethyl-2,3-dioleyloxy-1-propanaminium chloride (DOTMA) and the phospholipid 1,2-dioleoyl-sn-glycero-3-phosphoethanolamine (DOPE). In some embodiments, the pharmaceutical composition comprises at least one lipid. In some embodiments, the pharmaceutical composition comprises at least one cationic lipid. The cationic lipid can be monocationic or polycationic. Any cationic amphiphilic molecule, e.g., a molecule which comprises at least one hydrophilic and lipophilic moiety is a cationic lipid within the meaning of the present invention. In some embodiments, the positive charges are contributed by the at least one cationic lipid and the negative charges are contributed by the nucleic acid. In some embodiments, the pharmaceutical composition comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid. Examples of helper lipids include, but are not limited to, 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof.

In some embodiments, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In some embodiments, in this ratio, the molar amount of the cationic lipid results from the molar amount of the cationic lipid multiplied by the number of positive charges in the cationic lipid.

In some embodiments, the lipid is comprised in a vesicle encapsulating the vector genome. The vesicle may be a multilamellar vesicle, an unilamellar vesicle, or a mixture thereof. The vesicle may be a liposome.

### Vector Genomes

In some embodiments, the invention provides methods for determining an innate immune response to a gene therapy agent used for the delivery of a therapeutic transgene to a desired target in the individual. In some embodiments, the gene therapy agent comprises a vector genome for delivery and expression of the therapeutic transgene in the desired target in the individual.

The present invention contemplates the use of gene therapy agents for the introduction of one or more nucleic acid sequences encoding a therapeutic polypeptide and/or nucleic acid for packaging into a viral particle (for viral gene therapy agents). The vector genome may include any element to establish the expression of the therapeutic polypeptide and/or nucleic acid, for example, a promoter, an ITR of the present disclosure, a ribosome binding element, terminator, enhancer, selection marker, intron, polyA signal, and/or origin of replication.

In some embodiments, the therapeutic transgene encodes a therapeutic polypeptide. A therapeutic polypeptide may, e.g., supply a polypeptide and/or enzymatic activity that is absent or present at a reduced level in a cell or organism. Alternatively, a therapeutic polypeptide may supply a polypeptide and/or enzymatic activity that indirectly counteracts an imbalance in a cell or organism. For example, a therapeutic polypeptide for a disorder related to buildup of a metabolite caused by a deficiency in a metabolic enzyme or activity may supply a missing metabolic enzyme or activity, or it may supply an alternate metabolic enzyme or activity that leads to reduction of the metabolite. A therapeutic polypeptide may also be used to reduce the activity of a polypeptide (e.g., one that is overexpressed, activated by a gain-of-function mutation, or whose activity is otherwise misregulated) by acting, e.g., as a dominant-negative polypeptide.

The vector genomes of the invention may encode polypeptides that are intracellular proteins, anchored in the cell membrane, remain within the cell, or are secreted by the cell transduced with the vectors of the invention. For polypeptides secreted by the cell that receives the vector; the polypeptide can be soluble (i.e., not attached to the cell). For example, soluble polypeptides are devoid of a transmembrane region and are secreted from the cell. Techniques to identify and remove nucleic acid sequences which encode transmembrane domains are known in the art.

In some embodiments, the vector genome of the invention encodes polypeptides used to treat a disease or disorder in an individual. Diseases and disorders treated by the gene therapy agent of the invention include but are not limited to Huntington disease (HD), progressive supranuclear palsy (PSP), multiple system atrophy (MSA), metachromatic leukodystrophy (MLD), amyotrophic lateral sclerosis (ALS), age-related macular degeneration (AMD), congenital muscular dystrophy (CMD), phenylketonuria (PKU), muscular dystrophy (MD), A1AT deficiency, focal segmental glomerulosclerosis (FSGS), cystinuria, hemophilia A, hemophilia B, Gaucher disease (GBA), Parkinson's disease (PD), and Pompe disease.

In some embodiments, the therapeutic polypeptide is huntingtin (HTT), tau, amyloid precursor protein, alpha-synuclein, pseudoarylsulfatase (ARSA), superoxide dismutase 1 (SOD1), phenylalanine hydroxylase (PAH), dystrophin, alpha-1-antitrypsin (A1AT), cysteine transporter, Factor VIII (FVIII), Factor IX (FIX), acid beta-glucosidase, glial-derived growth factor (GDNF), brain-derived growth factor (BDNF), tyrosine hydroxlase (TH), GTP-cyclohydrolase (GTPCH), and/or amino acid decarboxylase (AADC), or alpha glucosidase.

In some embodiments, the heterologous nucleic acid encodes a therapeutic nucleic acid. In some embodiments, a therapeutic nucleic acid may include without limitation an siRNA, an shRNA, an RNAi, a miRNA, an antisense RNA, a ribozyme or a DNAzyme. As such, a therapeutic nucleic acid may encode an RNA that when transcribed from the nucleic acids of the vector can treat a disorder by interfering with translation or transcription of an abnormal or excess protein associated with a disorder of the invention. For example, the nucleic acids of the invention may encode for an RNA which treats a disorder by highly specific elimination or reduction of mRNA encoding the abnormal and/or excess proteins. Therapeutic RNA sequences include RNAi, small inhibitory RNA (siRNA), micro RNA (miRNA), and/or ribozymes (such as hammerhead and hairpin ribozymes) that can treat disorders by highly specific elimination or reduction of mRNA encoding the abnormal and/or excess proteins.

In some embodiments, the therapeutic polypeptide or therapeutic nucleic acid is used to treat a disorder of the CNS. Without wishing to be bound to theory, it is thought that a therapeutic polypeptide or therapeutic nucleic acid may be used to reduce or eliminate the expression and/or activity of a polypeptide whose gain-of-function has been associated with a disorder, or to enhance the expression and/or activity of a polypeptide to complement a deficiency that has been associated with a disorder (e.g., a mutation in a gene whose expression shows similar or related activity). Non-limiting examples of disorders of the invention that may be treated by a therapeutic polypeptide or therapeutic nucleic acid of the invention (exemplary genes that may be targeted or supplied are provided in parenthesis for each disorder) include stroke (e.g., caspase-3, Beclin1, Ask1, PAR1, HIF1α, PUMA, and/or any of the genes described in Fukuda, A. M. and Badaut, J. (2013) Genes (Basel) 4:435-456), Huntington's disease (mutant HTT), epilepsy (e.g., SCN1A, NMDAR, ADK, and/or any of the genes described in Boison, D. (2010) Epilepsia 51:1659-1668), Parkinson's disease (alpha-synuclein), Lou Gehrig's disease (also known as amyotrophic lateral sclerosis; SOD1), Alzheimer's disease (tau, amyloid precursor protein), corticobasal degeneration or CBD (tau), corticogasal ganglionic degeneration or CBGD (tau), frontotemporal dementia or FTD (tau), progressive supranuclear palsy or PSP (tau), multiple system atrophy or MSA (alpha-synuclein), cancer of the brain (e.g., a mutant or overexpressed oncogene implicated in brain cancer), and lysosomal storage diseases (LSD). Disorders of the invention may include those that involve large areas of the cortex, e.g., more than one functional area of the cortex, more than one lobe of the cortex, and/or the entire cortex. Other non-limiting examples of disorders of the invention that may be treated by a therapeutic polypeptide or therapeutic nucleic acid of the invention include traumatic brain injury, enzymatic dysfunction disorders, psychiatric disorders (including post-traumatic stress syndrome), neurodegenerative diseases, and cognitive disorders (including dementias, autism, and depression). Enzymatic dysfunction disorders include without limitation leukodystrophies (including Canavan's disease) and any of the lysosomal storage diseases described below.

In some embodiments, the therapeutic polypeptide or therapeutic nucleic acid is used to treat a lysosomal storage disease. As is commonly known in the art, lysosomal storage disease are rare, inherited metabolic disorders characterized by defects in lysosomal function. Such disorders are often caused by a deficiency in an enzyme required for proper mucopolysaccharide, glycoprotein, and/or lipid metabolism, leading to a pathological accumulation of lysosomally stored cellular materials. Non-limiting examples of lysosomal storage diseases of the invention that may be treated by a therapeutic polypeptide or therapeutic nucleic acid of the invention (exemplary genes that may be targeted or supplied are provided in parenthesis for each disorder) include Gaucher disease type 2 or type 3 (acid beta-glucosidase, GBA), GM1 gangliosidosis (beta-galactosidase-1, GLB1), Hunter disease (iduronate 2-sulfatase, IDS), Krabbe disease (galactosylceramidase, GALC), a mannosidosis disease (a mannosidase, such as alpha-D-mannosidase, MAN2B1), β mannosidosis disease (beta-mannosidase, MANBA), metachromatic leukodystrophy disease (pseudoarylsulfatase A, ARSA), mucolipidosisII/III disease (N-acetylglucosamine-1-phosphotransferase, GNPTAB), Niemann-Pick A disease (acid sphingomyelinase, ASM), Niemann-Pick C disease (Niemann-Pick C protein, NPC1), Pompe disease (acid alpha-1,4-glucosidase, GAA), Sandhoff disease (hexosaminidase beta subunit, HEXB), Sanfilippo A disease (N-sulfoglucosamine sulfohydrolase, MPS3A), Sanfilippo B disease (N-alpha-acetylglucosaminidase, NAGLU), Sanfilippo C disease (heparin acetyl-CoA:alpha-glucosaminidase N-acetyltransferase, MPS3C), Sanfilippo D disease (N-acetylglucosamine-6-sulfatase, GNS), Schindler disease (alpha-N-acetylgalactosaminidase, NAGA), Sly disease (beta-glucuronidase, GUSB), Tay-Sachs disease (hexosaminidase alpha subunit, HEXA), and Wolman disease (lysosomal acid lipase, LIPA).

In some embodiments, the therapeutic polypeptide encodes Factor VIII, Factor IX, myotubularin, survival motor neuron protein (SMN), retinoid isomerohydrolase (RPE65), NADH-ubiquinone oxidoreductase chain 4, Choroideremia protein (CHM), ornithine transcarbomylase, argininosuccinate synthetase, β-globin, γ-globin, phenylalanine hydroxylase, adrenoleukodystrophy protein (ALD), dystrophin, a truncated dystrophin, an anti-VEGF agent, or a functional variant thereof.

In some embodiments, the heterologous nucleic acid is operably linked to a promoter. Exemplary promoters include, but are not limited to, the cytomegalovirus (CMV) immediate early promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 108(2):193-9) and the elongation factor 1-alpha promoter (EF1-alpha) promoter (Kim et al., Gene, 1990, 91(2):217-23 and Guo et al., Gene Ther., 1996, 3(9):802-10). In some embodiments, the promoter comprises a human β-glucuronidase promoter or a cytomegalovirus enhancer linked to a chicken β-actin (CBA) promoter. The promoter can be a constitutive, inducible or repressible promoter. In some embodiments, the invention provides a recombinant vector comprising nucleic acid encoding a heterologous transgene of the present disclosure operably linked to a CBA promoter. Exemplary promoters and descriptions may be found, e.g., in U.S. PG Pub. 20140335054.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the 13-actin promoter, the phosphoglycerol kinase (PGK) promoter, and the EFla promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter, or fragment thereof, for the transgene will be used. The native promoter can be used when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art.

In some embodiments, the vector comprises an intron. For example, in some embodiments, the intron is a chimeric intron derived from chicken beta-actin and rabbit beta-globin. In some embodiments, the intron is a minute virus of mice (MVM) intron.

In some embodiments, the vector comprises a polyadenylation (polyA) sequence. Numerous examples of polyadenylation sequences are known in the art, such as a bovine growth hormone (BGH) Poly(A) sequence (see, e.g., accession number EF592533), an SV40 polyadenylation sequence, and an HSV TK pA polyadenylation sequence.

### Kits and Articles of Manufacture

The gene therapy agent (e.g., an AAV particle, an adenovirus particle, a lentivirus particle, a HSV particle, or a lipid nanoparticle) and/or materials for isolating dendritic cells as described herein may be contained within a kit or article of manufacture, e.g., designed for use in one of the methods of the invention as described herein.

In some embodiments, the kits or articles of manufacture further include instructions for assaying innate immunogenicity to the gene therapy agent using isolated dendritic cells. The kits or articles of manufacture described herein may further include other materials desirable from a commercial and user standpoint, including other buffers, diluents, filters, needles, syringes, and package inserts with instructions for performing any methods described herein. Suitable packaging materials may also be included and may be any packaging materials known in the art, including, for example, vials (such as sealed vials), vessels, ampules, bottles, jars, flexible packaging (e.g., sealed Mylar or plastic bags), and the like. These articles of manufacture may further be sterilized and/or sealed.

In some embodiments, the kits or articles of manufacture further contain one or more of the buffers and/or pharmaceutically acceptable excipients described herein (e.g., as described in REMINGTON'S PHARMACEUTICAL SCIENCES (Mack Pub. Co., N.J. 1991). In some embodiments, the kits or articles of manufacture include one or more pharmaceutically acceptable excipients, carriers, solutions, and/or additional ingredients described herein. The kits or articles of manufacture described herein can be packaged in single unit dosages or in multidosage forms. The contents of the kits or articles of manufacture are generally formulated as sterile and can be lyophilized or provided as a substantially isotonic solution.

### Exemplary Embodiments

The Invention includes the following enumerated embodiments.

1. A method of determining the innate immunogenicity to a gene therapy agent in an individual, the method comprising
a) incubating an innate immune cell from the individual with the gene therapy agent,
b) analyzing the innate immune cell for altered expression of one or more cytokines compared to a suitable control, whereby altered expression of the one or more cytokines produces a cytokine signature,
wherein expression of a cytokine signature following incubation with the gene therapy agent indicates innate immunogenicity to the gene therapy agent in the individual.

2. The method of embodiment 1, wherein the innate immune cell is a dendritic cell, a monocyte, a macrophage or a natural killer (NK) cell.

3. The method of embodiment 1 or 2, wherein the innate immune cell is isolated from peripheral blood mononuclear cells from the individual.

4. The method of any one of embodiments 1-3, wherein the innate immune cell is a dendritic cell.

5. The method of embodiment 4, wherein the dendritic cell is derived from a monocyte of the individual.

6. The method of embodiment 4 or 5, further comprising isolating monocytes from the individual and incubating the monocytes in dendritic cell culture media to derive dendritic cells from the monocytes prior to incubating the dendritic cells with the gene therapy agent.

7. The method of embodiment 6, wherein the monocytes are CD14+ monocytes.

8. The method of embodiment 6 or 7, wherein the monocytes are incubated with the dendritic cell culture media for about 5 to about 10 days or about 7 to about 8 days to derive dendritic cells from the monocytes.

9. The method of any one of embodiments 1-8, wherein the innate immune cells are replated prior to the incubation with the gene therapy agent of step b).

10. The method of embodiment 9, wherein the innate immune cells are replated into microwell dishes.

11. The method of any one of embodiments 1-10, wherein the gene therapy agent is a viral vector and wherein the innate immune cells are incubated with the gene therapy agent at an MOI of about 1×10³ to about 1×10⁵ or about 1×10⁴.

12. The method of any one of embodiments 1-10, wherein the gene therapy agent is a non-viral vector, and wherein the innate immune cells are incubated with the non-viral vector at a concentration of about 1 ng/mL to about 1 mg/mL.

13. The method of any one of embodiments 1-12, wherein the innate immune cells are incubated with the gene therapy agent for about 12 hours to about 36 hours or about 24 hours.

14. The method of any one of embodiments 1-13, wherein the cytokine signature comprises increased expression of IL6, TNFα, and IL-1β.

15. The method of any one of embodiments 1-14, wherein the cytokine signature comprises increased expression of one or more of IL6, TNFα, IL-1β, MCP1 and MIP-1 a.

16. The method of any one of embodiments 1-15, wherein the cytokine signature comprises increased expression of IL6, TNFα, IL-1β, MCP1 and MIP-1α.

17. The method of any one of embodiments 1-15, wherein the cytokine signature comprises increased expression of IL6, TNFα, and IL-1β.

18. The method of any one of embodiments 1-17, wherein expression of the cytokines in the cytokine signature is increased compared to a suitable control.

19. The method of embodiment 18, wherein the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

20. The method of any one of embodiments 1-10 and 13-19, wherein the gene therapy agent is a viral vector.

21. A method of determining the innate immunogenicity to a viral gene therapy agent in an individual, the method comprising
a) incubating monocytes from the individual in dendritic cell culture media under conditions in which the monocytes differentiate into dendritic cells,
b) incubating the dendritic cells with the viral gene therapy agent at an MOI of about 1×10³ to about 1×10⁵ for about 12 to about 36 hours,
c) analyzing the dendritic cells for altered expression of one or more cytokines compared to a suitable control, whereby altered expression of the one or more cytokines produces a cytokine signature,
wherein expression of the cytokine signature following incubation with the viral gene therapy agent indicates innate immunogenicity to the viral gene therapy agent in the individual, wherein the cytokine signature comprises increased expression of IL6, TNFα, and IL-1β.

22. The method of embodiment 21, wherein the monocytes are obtained from peripheral mononuclear cells from the individual.

23. The method of embodiment 21 or 22, wherein the monocytes are CD14+ monocytes.

24. The method of any one of embodiments 21-23, wherein the monocytes are incubated in dendritic cell culture media for about 7-8 days to differentiate the monocytes to dendritic cells.

25. The method of any one of embodiments 21-24, wherein the dendritic cells are incubated with the viral gene therapy agent at an MOI of about 1×10⁴

26. The method of any one of embodiments 21-25, wherein the dendritic cells are incubated with the viral gene therapy agent for about 24 hours

27. The method of any one of embodiments 20-26, wherein the viral vector is an AAV particle.

28. The method of embodiment 27, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LKO3 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

29. The method of embodiment 28, wherein the AAV capsid comprises a tyrosine mutation, a heparin binding mutation, or an HBKO mutation.

30. The method of any one of embodiments 27-29, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

31. The method of embodiment 30, wherein the one or more ITRs and the capsid of the AAV particle are derived from the same AAV serotype.

32. The method of embodiment 30, wherein the one or more ITRs and the capsid of the AAV particles are derived from different AAV serotypes.

33. The method of any one of embodiments 20-26, where the viral vector is an adenoviral particle.

34. The method of embodiment 33, wherein the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

35. The method of any one of embodiments 20-26, where the viral vector is a lentiviral particle.

36. The method of embodiment 35, wherein the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

37. The method of any one of embodiments 20-26, where the viral vector is a Herpes simplex virus (HSV) particle.

38. The method of embodiment 37, wherein the HSV particle is an HSV-1 particle or an HSV-2 particle, or a functional variant thereof.

39. The method of any one of embodiments 1-10 and 12-19, wherein the gene therapy agent is a lipid nanoparticle.

40. A method of determining the innate immunogenicity to a non-viral gene therapy agent in an individual, the method comprising
a) incubating monocytes from the individual in dendritic cell culture media under conditions in which the monocytes differentiate into dendritic cells,
b) incubating the dendritic cells with the non-viral vector at a concentration of about 1 ng/mL to about 1 mg/mL,
c) analyzing the dendritic cells for altered expression of one or more cytokines compared to a suitable control, whereby altered expression of the one or more cytokines produces a cytokine signature,
wherein expression of the cytokine signature following incubation with the non-viral gene therapy agent indicates innate immunogenicity to the non-viral gene therapy agent in the individual, wherein the cytokine signature comprises increased expression of IL6, TNFα, and

41. The method of embodiment 40, wherein the monocytes are obtained from peripheral mononuclear cells from the individual.

42. The method of embodiment 40 or 41, wherein the monocytes are CD14+ monocytes.

43. The method of any one of embodiments 40-42, wherein the monocytes are incubated in dendritic cell culture media for about 7-8 days to differentiate the monocytes to dendritic cells.

44. The method of any one of embodiments 40-43, wherein the dendritic cells are incubated with the non-viral gene therapy agent for about 12 hours to about 36 hours or about 24 hours

45. The method of any one of embodiments 1-44, wherein the gene therapy agent comprises nucleic acid encoding a heterologous transgene.

46. The method of embodiment 45, wherein the heterologous transgene is operably linked to a promoter.

47. The method of embodiment 46, wherein the promoter is a constitutive promoter, a tissue-specific promoter, or an inducible promoter.

48. A method of determining a cytokine signature of a gene therapy agent comprising
a) incubating one or more innate immune cell from one or more individuals with the gene therapy agent,
b) analyzing the one or more innate immune cells for altered expression of one or more cytokines compared to a suitable control,
wherein the altered expression of one or more cytokines in step b) indicates the cytokine signature of the gene therapy agent.

49. The method of embodiment 48, wherein the innate immune cell is a dendritic cell, a monocyte, a macrophage or a natural killer (NK) cell.

50. The method of embodiment 48 or 49, wherein the one or more innate immune cells are isolated from peripheral blood mononuclear cells from the individual.

51. The method of any one of embodiments 48-50, wherein the innate immune cell is a dendritic cell.

52. The method of embodiment 51, wherein the dendritic cell is derived from a monocyte of the one or more individuals.

53. The method of embodiment 51 or 52, further comprising isolating monocytes from the one or more individuals and incubating the monocytes in dendritic cell culture media to derive dendritic cells from the monocytes prior to incubating the dendritic cells with the gene therapy agent.

54. The method of embodiment 53 wherein the monocytes are CD14+ monocytes.

55. The method of embodiment 53 or 54, wherein the monocytes are incubated with the dendritic cell culture media for about 5 to about 10 days or about 7 to about 8 days to derive dendritic cells from the monocytes.

56. The method of any one of embodiments 48-55, wherein the innate immune cells are replated prior to the incubation with the gene therapy agent of step b).

57. The method of any one of embodiments 48-56, wherein the gene therapy agent is a viral vector and wherein the innate immune cells are incubated with the gene therapy agent at an MOI of about 1×10³ to about 1×10⁵ or about 1×10⁴.

58. The method of any one of embodiments 48-56, wherein the gene therapy agent is a non-viral vector, and wherein the innate immune cells are incubated with the non-viral vector at a concentration of about 1 ng/mL to about 1 mg/mL.

59. The method of any one of embodiments 48-58, wherein the innate immune cells are incubated with the gene therapy agent for about 12 hours to about 36 hours or about 24 hours.

60. The method of any one of embodiments 48-59, wherein expression of the cytokines in the cytokine signature is increased compared to a suitable control.

61. The method of embodiment 60, wherein the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

62. The method of any one of embodiments 48-57 and 59-61, wherein the gene therapy agent is a viral vector.

63. The method of embodiment 57 or 62, wherein the viral vector is an AAV particle.

64. The method of embodiment 63, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LKO3 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

65. The method of embodiment 64, wherein the AAV capsid comprises a tyrosine mutation, a heparin binding mutation, or an HBKO mutation.

66. The method of any one of embodiments 63-65, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

67. The method of embodiment 66, wherein the one or more ITRs and the capsid of the AAV particle are derived from the same AAV serotype.

68. The method of embodiment 66, wherein the one or more ITRs and the capsid of the AAV particles are derived from different AAV serotypes.

69. The method of embodiment 57 or 62, wherein the viral vector is an adenoviral particle.

70. The method of embodiment 69, wherein the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

71. The method of embodiment 57 or 62, where the viral vector is a lentiviral particle.

72. The method of embodiment 71, wherein the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

73. The method of embodiment 57 or 62, where the viral vector is a Herpes simplex virus (HSV) particle.

74. The method of embodiment 73, wherein the HSV particle is an HSV-1 particle or an HSV-2 particle, or a functional variant thereof.

75. The method of any one of embodiments 48-56 and 58-61, wherein the gene therapy agent is a lipid nanoparticle.

76. The method of any one of embodiments 48-75, wherein the gene therapy agent comprises nucleic acid encoding a heterologous transgene.

77. The method of embodiment 76, wherein the heterologous transgene is operably linked to a promoter.

78. The method of embodiment 77, wherein the promoter is a constitutive promoter, a tissue-specific promoter, or an inducible promoter.

79. A kit for use in the methods of any one of embodiments 1-78.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Screening Innate Immunogenicity of Different AAV Vectors

This example provides a strategy for assaying the innate immune response following AAV treatment.

Immune responses to AAV vectors pose challenges to successful clinical application. AAVs trigger an immune response involving activation of both the innate and adaptive immune systems. While the adaptive immune response to AAV is relatively well characterized, innate immune activation by AAV is poorly understood.

### Materials and Methods

Preparation of Peripheral Blood Mononuclear Cells. Blood from leukopaks (AllCells) from four donors was decanted into 50 mL tubes and Dulbecco's phosphate buffered saline (DPBS) was added at a 1:1 ratio. The blood plus DBPS mix was gently pipetted into a separate 50 mL tube containing 15 mL Ficoll (GE17-5442-02) to ensure no mixing of the blood and Ficoll phases. The mixture was centrifuged at 2000 RPM for 25 minutes at room temperature (9 acceleration and no braking). The buffy coat containing the peripheral blood mononuclear cells (PBMCs) was collected, transferred to a new tube, and centrifuged for five minutes at 400 RCF. PBMCs were washed three times with phosphate buffered saline (PBS) containing 1% fetal bovine serum (FBS) or fetal calf serum (FCS) and counted.

Isolation of Monocytes. CD14+ monocytes were isolated from the PBMCs using CD14 MicroBeads following the manufacturer's protocol (Milteny Biotech, Germany, order no. 130-050-201, protocol available online world wide web miltenyibiotec.com/upload/assets/IM0001260.PDF). Briefly, PBMCs were incubated with 20 μL of CD14 MicroBeads per 10⁷ total cells for 15 minutes at 2-8° C. PBMCs were applied onto a magnetic column (Miltenyi; world wide web miltenyibiotec.com/US-en/products/ls-columns.html #130-042-401) and unlabeled cells were allowed to pass through. After washing the column three times, the column was removed from the magnetic separator, (Miltneyi; world wide web miltenyibiotec.com/US-en/products/quadromacs-separator-and-starting-kits.html #130-091-051) placed on a collection tube, and magnetically labeled CD14+ monocytes were flushed out by pushing a plunger into the column.

Differentiation of Monocytes. CD14+ monocytes were differentiated into dendritic cells using ImmunoCult-ACF Dendritic Cell Media, Differentiation Supplement, and Maturation Supplement (Stem Cell Technologies, catalog #10986, #10988, and #10989; world wide web cdn.stemcell.com/media/Files/pis/DX20521-PIS_1_2_0.pdf?_ga=2.81451927.1035383195.1642105700-1174975582.1603298321) following the manufacturer's protocol available online. Briefly, purified CD14+ monocytes were added to Dendritic Cell Media containing the Differentiation Supplement and incubated at 37° C. for three days. On day 3, media was replaced with fresh Dendritic Cell media containing Differentiation Supplement and cells were incubated for two additional days. On day 5, Maturation Supplement was added to the cells at a 1 to 100 dilution (e.g. 50 μL supplement per 5 mL culture). Differentiated dendritic cells were harvested on day 7.

rAAV Production and Titration. rAAV vectors (AAV1, AAV2, AAVDJ and AAVrh32.33) were produced using a standard triple transfection method (Sena-Esteves and Gao, Cold Spring Harb Protoc; doi:10.1101/pdb.top095513, 2020). All serotypes tested encode the same GFP transgene. Viruses were purified by cesium chloride ultracentrifugation and titrated using both silver staining and quantitative polymerase chain reaction (qPCR).

### Treatments

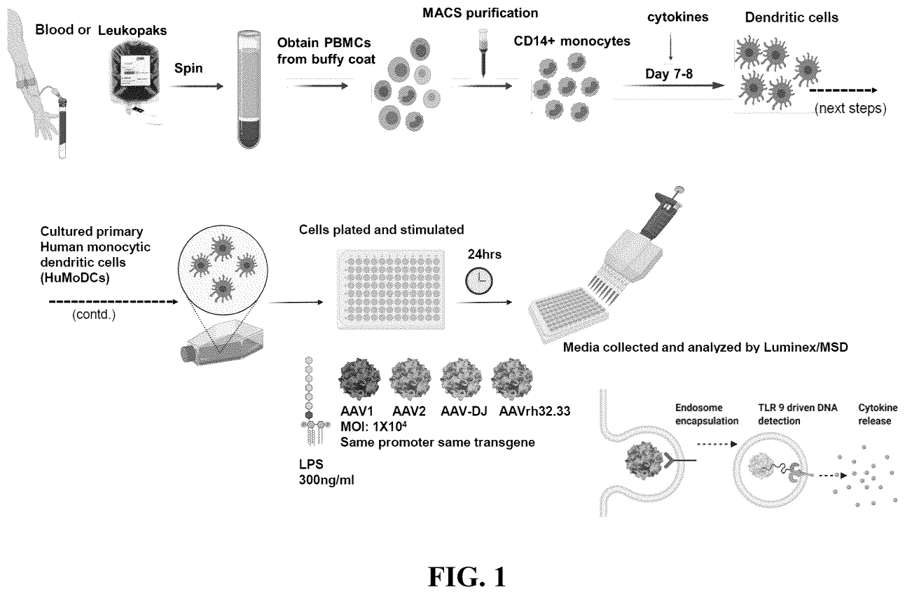

Dendritic cells were plated in 96 well plates with 200,000 cells per well. Each treatment was performed in triplicates. Four different treatments with four different AAV vectors at 1e4 MOI were used. The cells were incubated at 37° C., 5% CO₂ in an incubator for 24 hours. After 24 hrs, plates were centrifuged and media supernatant was collected. ipopolysaccharide LPS (300 ng/mL) (Sigma Aldrich Fine Chemicals Biosciences L2630100MG) was used as a positive control which is known to activate toll like receptor 4 and induce cytokine production I human monocytic derived dendritic cells. Cell media was then collected and centrifuged. The clarified media was analyzed by two different methods to detect different cytokines released from dendritic cells upon challenge with different AAV serotypes. The two methodologies used Multiplex Immunoassay (Luminex Bead-based assay) performed by Nanobiotec (world wide web nanobiotecusa.com/immunoassay and MSD (world wide web mesoscale.com/products/v-plex-proinflammatory-panel-1-human-kit-k15049W) performed by DC₃ therapeutics https://www.dc3therapeutics.com/services. The experiment outline of PBMC isolation, monocyte purification and dendritic cell differentiation is illustrated in FIG. 1. This figure also shows how dendritic cells were treated with different AAVs.

### Results

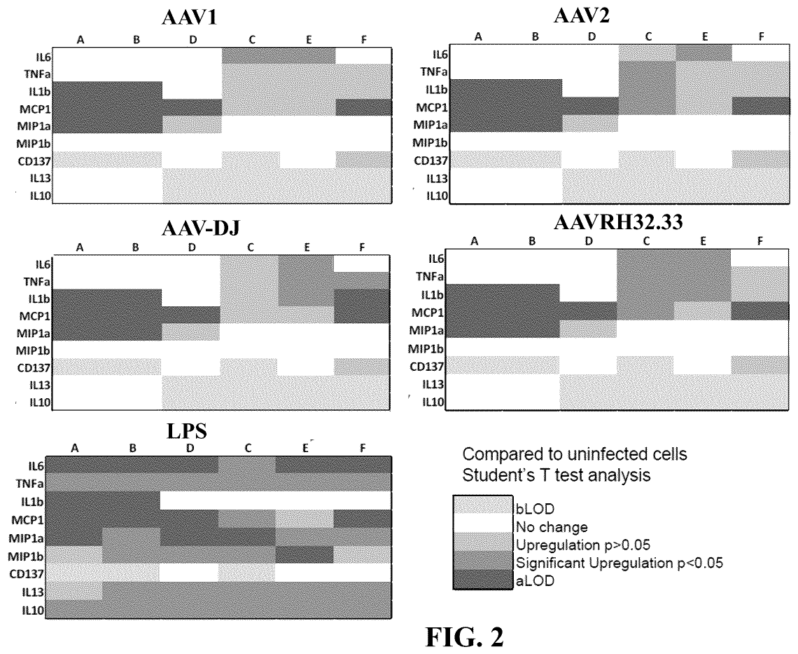

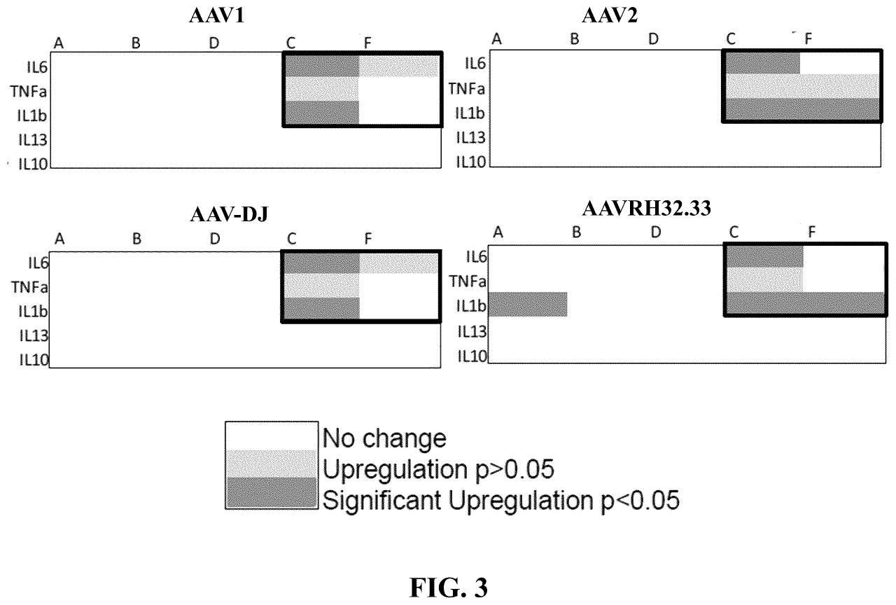

All four serotypes tested produced a weaker immune response than LPS based on cytokines released by the treated dendritic cells (FIG. 2). While LPS induces a significant upregulation in the majority of cytokines assayed from each donor, the different AAV serotypes tested induce a less pronounced upregulation that is limited to only a subset of cytokines, IL6, TNFα, IL-1β, MCP1 and MIP-1α (FIG. 2). Notably, this subset of cytokines (i.e., cytokine signature) appear to be conserved across different AAV serotypes. The data presented in FIG. 2 is based on Luminex Technology where some cytokines are above or below the limit of detection (aLOD/bLOD) therefore we utilized MSD technology and examined cytokine levels from the same donor cells again. As seen in FIG. 2, the same results were verified using an orthogonal MSD assay (FIG. 3).

Of the AAV serotypes tested, AAVrh32.33 was found to generate the most robust and consistent innate immune signature (FIGS. 2 and 3). This was evidenced by the greater upregulation observed for IL-6, TNF-α, and IL-1β.

### Discussion

Success of gene therapy for treatment of rare genetic diseases relies heavily on adeno-associated virus (AAV) viral vectors that provide many attractive features including tissue specific tropism, transduction of quiescent cells and maintenance of modified gene expression. However, immune responses to AAV vectors pose a major challenge for successful clinical translation. The capsid, viral genome as well as transgene trigger immune responses that involve activation of both innate and adaptive arms of the immune system. The adaptive immune responses triggered by B and T cells have been understood in the field to some extent, the innate immune activation is very poorly understood. Studies based on mouse models indicate that TLR9a DNA sensor present in the endosome of the cells detects AAV genomes and activates a signaling cascade that ultimately results in cytokine release (Ashley, S N et al., Cell Immunol. 2019, 346:103997; (Zhu, J et al., J Clin Invest. 2009; 119(8):2388-2398). (FIG. 1, Bottom right diagram). These cytokines mount an anti-viral response and trigger activation of adaptive immune systems. A major challenge in understanding these innate immune responses lies in the poor reproducibility of immune responses observed in clinical trials to an ex-vivo setting. To circumvent this challenge and understand innate immune responses we have now developed a novel assay that recapitulates innate immune signature in various human donors in response to various AAV serotypes. We have used human monocytic dendritic cells (moDCs) from healthy donors and we sensitively detect the cytokines released from these moDCs upon challenge with AAV vectors.

### Example 2: Screening Innate Immunogenicity of LNPs

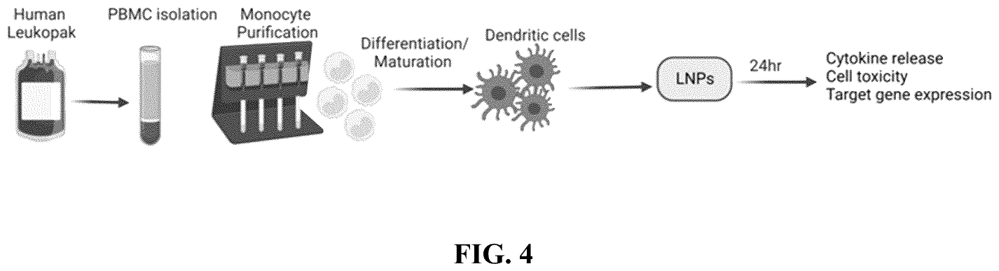

This example provides a strategy for assaying the innate immune response following LNP delivery. A general schematic of the process described in the Example is depicted in FIG. 4.

### Materials and Methods

### Production of Differentiated Dendritic Cells

Blood mononuclear cells were prepared and monocytes were isolated and differentiated into dendritic cells using the procedure described in Example 1.

### LNP Treatments and Cytokine Measurement

Human monocytic dendritic cells were plated in 96 well format and treated with 10 ug LNPs at 37° C. using standard protocols (see https://www.ncbi.nlm.nih.gov/pmc/articles/PMC5577173/and https://www.ncbi.nlm.nih.gov/pmc/articles/PMC5646367/) and harvested 24 hours later for downstream analysis such as cell cytotoxicity and target gene expression via flow cytometry. The media was collected for cytokine analysis. The cells were centrifugated at 2,000 rpm for 5 minutes, and the supernatants were collected to analyze cytokines. Cytokines were measured using luminex using the MILLIPLEX® Human Cytokine/Chemokine/Growth Factor Panel A 38 Plex Premixed Magnetic Bead Panel—Immunology Multiplex Assay Catalog #HCYTA-60K-PX38 following manufacturer's protocol.

Reagents used in the assays are shown in Table 1.

TABLE 1
List of Reagents and Suppliers

| Reagents | Catalog number | Supplier |
|---|---|---|
| Biotinylated Human Siglec- | SI2-H82E3-200ug | Acrobiosystems Inc |
| 2/CD22 Protein, | | |
| Fc, Avitag ™ | | |
| CD22 Monoclonal Antibody | 17022942 | Fisher Scientific |
| (eBio4KB128 (4KB128)), | | |
| APC, eBioscience ™, | | |
| Invitrogen ™ | | |
| LIVE/DEAD ™ Fixable | L34975 | Invitrogen |
| Near-IR Dead Cell Stain | | |
| Kit, for 633 or 635 nm | | |
| excitation | | |
| Pacific Blue ™ anti-human | 337212 | Biolegend |
| CD11c Antibody | | |
| FITC anti-human CD11b | 301330 | Biolegend |
| Antibody | | |
| PE anti-human CD83 | 305308 | Biolegend |
| Antibody | | |
| Dulbecco's Phosphate | 7905 | Stem cells |
| Buffered Saline with 2% | | technologies |
| Fetal | | |
| Bovine Serum (FACS | | |
| buffer) | | |

### Cell Viability Assessment

Dendritic cells (DCs) were resuspended in the FACS buffer and transferred to a U-bottom 96-well plate. The cells were centrifuged at 2,000 rpm for 5 minutes, and the supernatant was discarded. To analyze the cell viability, Live/Dead negative staining cells were gated, and the percentage was determined to be the cell viability. LIVE/DEAD™ Fixable Near-IR Dead Cell Stain Kit, for 633 or 635 nm excitation Catalog #L34975 from Invitrogen was used for cell staining.

### Target Gene Expression by Flow Cytometry

Dendritic cells (DCs) were resuspended in the FACS buffer and transferred to a U-bottom 96-well plate. The cells were centrifuged at 2,000 rpm for 5 minutes, and the supernatant was discarded. To analyze target gene expression CD22 protein was diluted with the FACS buffer at a final concentration of 5 μg/ml final concentration. DCs in each well was treated with 100 μl of the diluted CD22 for 30 minutes at 4° C. Cells were washed with the FACS buffer and then stained with 50 μl of flow antibody master mix (1:100 anti-hCD22 APC, 1:100 anti-hCD11c Pacific Blue, 1:100 anti-hCD11b FITC, 1:100 anti-hCD83 PE, and 1:100 Live/Dead staining APC-Cy7) for 30 minutes at 4° C. The cells were washed with the FACS buffer 2 times, and the cells were run with a flow cytometer (Novocyte Penteon Flow Cytometer Systems 5 Lasers, Agilent Technology).

### Results

### Cell Viability

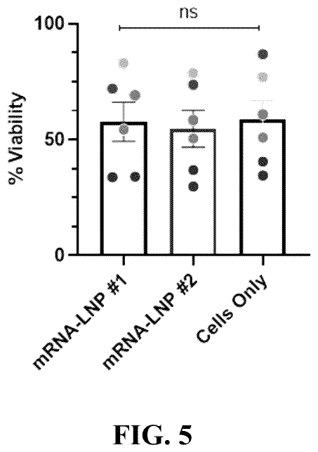

Dendritic cells were harvested post 24 hours after LNP treatments and flow cytometry was performed to assess cell viability (FIG. 5). Each dot in FIG. 5 represents cells derived from a human donor. The cells only condition received no LNP treatment and mRNA-LNP #1 and mRNA-LNP #2 were cells treated with two different LNPs, for all the donors the viability was same as the cells only control indicating no cell toxicity upon LNP treatments. The dendritic cell system can be used to assess LNPs without impacting cellular viability. One way ANOVA was performed to measure statistical significance.

### Transduction Levels

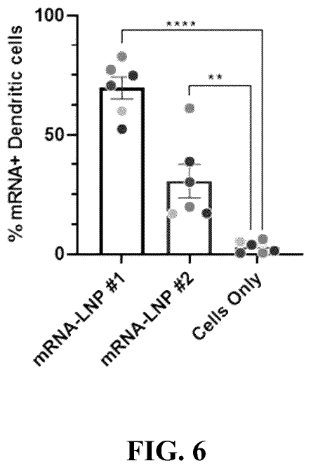

Dendritic cells were harvested post 24 hours after LNP treatments and flow cytometry was performed to assess target gene expression (FIG. 6). Each dot in FIG. 6 represents cells derived from a human donor. The cells only condition received no LNP treatment and mRNA-LNP #1 and mRNA-LNP #2 were cells treated with two different LNPs that encapsulated the same mRNA. Dendritic cells from all the different donors were transduced by LNPs as measured by the percentage of mRNA expressing cells. One way ANOVA was performed to measure statistical significance. This indicates that the disclosed dendritic cell assay system can be used to assess LNP transduction effectively.

### LNP Immunogenicity

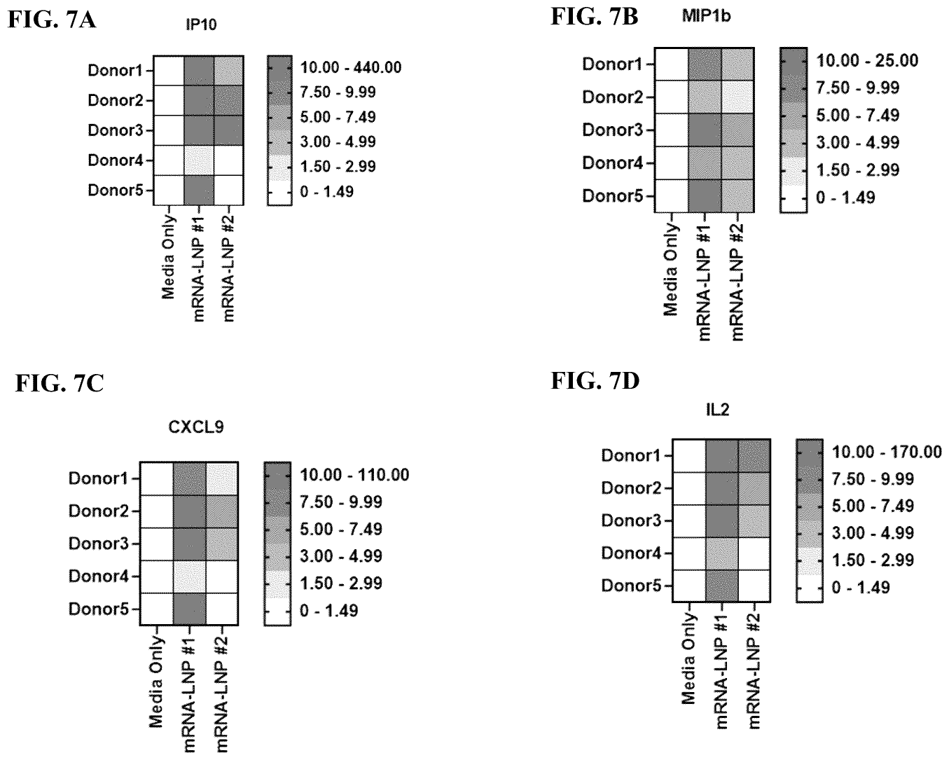

Media from dendritic cells was collected post 24 hours after LNP treatments and Luminex analysis was performed to identify the cytokine signature. mRNA encapsulating LNPs specifically secrete cytokines as compared to cells that had no LNP treatment. The analysis shows that cytokines such as IP10 (FIG. 7A), MIP1b (FIG. 7B), CXCL9 (FIG. 7C) and IL2 (FIG. 7D) that were upregulated across all donors as compared to respective media only controls.

What is claimed is:

1. A method of determining the innate immunogenicity to a gene therapy agent in an individual, the method comprising
a) incubating an innate immune cell from the individual with the gene therapy agent,
b) analyzing the innate immune cell for altered expression of one or more cytokines compared to a suitable control, whereby altered expression of the one or more cytokines produces a cytokine signature,
wherein expression of a cytokine signature following incubation with the gene therapy agent indicates innate immunogenicity to the gene therapy agent in the individual.

2. The method of claim 1, wherein the innate immune cell is a dendritic cell, a monocyte, a macrophage or a natural killer (NK) cell.

3. The method of claim 1 or 2, wherein the innate immune cell is isolated from peripheral blood mononuclear cells from the individual.

4. The method of any one of claims 1-3, wherein the innate immune cell is a dendritic cell.

5. The method of claim 4, wherein the dendritic cell is derived from a monocyte of the individual.

6. The method of claim 4 or 5, further comprising isolating monocytes from the individual and incubating the monocytes in dendritic cell culture media to derive dendritic cells from the monocytes prior to incubating the dendritic cells with the gene therapy agent.

7. The method of claim 6, wherein the monocytes are CD14+ monocytes.

8. The method of claim 6 or 7, wherein the monocytes are incubated with the dendritic cell culture media for about 5 to about 10 days or about 7 to about 8 days to derive dendritic cells from the monocytes.

9. The method of any one of claims 1-8, wherein the innate immune cells are replated prior to the incubation with the gene therapy agent of step b).

10. The method of claim 9, wherein the innate immune cells are replated into microwell dishes.

11. The method of any one of claims 1-10 wherein the gene therapy agent is a viral vector or a non-viral vector.

12. The method of claim 11 wherein the viral vector is an AAV.

13. The method of claim 11 wherein the non-viral vector is a lipid nanoparticle (LNP).

14. The method of any one of claims 1-10, wherein the gene therapy agent is a viral vector and wherein the innate immune cells are incubated with the gene therapy agent at an MOI of about 1×10³ to about 1×10⁵ or about 1×10⁴.

15. The method of any one of claims 1-10, wherein the gene therapy agent is a non-viral vector, and wherein the innate immune cells are incubated with the non-viral vector at a concentration of about 1 ng/mL to about 1 mg/mL.

16. The method of any one of claims 1-15, wherein the innate immune cells are incubated with the gene therapy agent for about 12 hours to about 36 hours or about 24 hours.

17. The method of any one of claims 1-16, wherein the cytokine signature comprises increased expression of IL6, TNFα, and IL-1β.

18. The method of any one of claims 1-17, wherein the cytokine signature comprises increased expression of one or more of IL6, TNFα, IL-1β, MCP1 and MIP-1α.

19. The method of any one of claims 1-18, wherein the cytokine signature comprises increased expression of IL6, TNFα, IL-1β, MCP1 and MIP-1α.

20. The method of any one of claims 1-18, wherein the cytokine signature comprises increased expression of IL6, TNFα, and IL-1β.

21. The method of any one of claims 1-16, wherein the cytokine signature comprises increased expression of one or more of IP10, MIP1b, CXCL9, and IL2.

22. The method of any one of claims 1-16, wherein the cytokine signature comprises increased expression of IP10, MIP1b, CXCL9, and IL2.

23. The method of any one of claims 1-22, wherein expression of the cytokines in the cytokine signature is increased compared to a suitable control.

24. The method of claim 23, wherein the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

25. The method of any one of claims 1-13 and 16-24, wherein the gene therapy agent is a viral vector.

26. A method of determining the innate immunogenicity to a viral gene therapy agent in an individual, the method comprising
a) incubating monocytes from the individual in dendritic cell culture media under conditions in which the monocytes differentiate into dendritic cells,
b) incubating the dendritic cells with the viral gene therapy agent at an MOI of about 1×10³ to about 1×10⁵ for about 12 to about 36 hours,
c) analyzing the dendritic cells for altered expression of one or more cytokines compared to a suitable control, whereby altered expression of the one or more cytokines produces a cytokine signature,
wherein expression of the cytokine signature following incubation with the viral gene therapy agent indicates innate immunogenicity to the viral gene therapy agent in the individual, wherein the cytokine signature comprises increased expression of IL6, TNFα, and IL-1β.

27. The method of claim 26, wherein the monocytes are obtained from peripheral mononuclear cells from the individual.

28. The method of claim 26 or 27, wherein the monocytes are CD14+ monocytes.

29. The method of any one of claims 26-28, wherein the monocytes are incubated in dendritic cell culture media for about 7-8 days to differentiate the monocytes to dendritic cells.

30. The method of any one of claims 26-29, wherein the dendritic cells are incubated with the viral gene therapy agent at an MOI of about 1×10⁴

31. The method of any one of claims 26-30, wherein the dendritic cells are incubated with the viral gene therapy agent for about 24 hours

32. The method of any one of claims 25-31, wherein the viral vector is an AAV particle.

33. The method of claim 32, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LKO3 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

34. The method of claim 33, wherein the AAV capsid comprises a tyrosine mutation, a heparin binding mutation, or an HBKO mutation.

35. The method of any one of claims 32-34, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

36. The method of claim 35, wherein the one or more ITRs and the capsid of the AAV particle are derived from the same AAV serotype.

37. The method of claim 35, wherein the one or more ITRs and the capsid of the AAV particles are derived from different AAV serotypes.

38. The method of any one of claims 25-31, wherein the viral vector is an adenoviral particle.

39. The method of claim 38, wherein the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

40. The method of any one of claims 25-31, where the viral vector is a lentiviral particle.

41. The method of claim 40, wherein the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

42. The method of any one of claims 25-31, where the viral vector is a Herpes simplex virus (HSV) particle.

43. The method of claim 42, wherein the HSV particle is an HSV-1 particle or an HSV-2 particle, or a functional variant thereof.

44. The method of any one of claims 1-10 and 15-20, wherein the gene therapy agent is a lipid nanoparticle.

45. The method of claim 21 or claim 22, wherein the gene therapy agent is a lipid nanoparticle.

46. A method of determining the innate immunogenicity to a non-viral gene therapy agent in an individual, the method comprising
a) incubating monocytes from the individual in dendritic cell culture media under conditions in which the monocytes differentiate into dendritic cells,
b) incubating the dendritic cells with the non-viral vector at a concentration of about 1 ng/mL to about 1 mg/mL,
c) analyzing the dendritic cells for altered expression of one or more cytokines compared to a suitable control, whereby altered expression of the one or more cytokines produces a cytokine signature,
wherein expression of the cytokine signature following incubation with the non-viral gene therapy agent indicates innate immunogenicity to the non-viral gene therapy agent in the individual, wherein the cytokine signature comprises increased expression of IL6, TNFα, and IL-1β.

47. The method of claim 46, wherein the monocytes are obtained from peripheral mononuclear cells from the individual.

48. The method of claim 46 or 47, wherein the monocytes are CD14+ monocytes.

49. The method of any one of claims 46-48, wherein the monocytes are incubated in dendritic cell culture media for about 7-8 days to differentiate the monocytes to dendritic cells.

50. The method of any one of claims 46-49, wherein the dendritic cells are incubated with the non-viral gene therapy agent for about 12 hours to about 36 hours or about 24 hours

51. The method of any one of claims 1-50, wherein the gene therapy agent comprises nucleic acid encoding a heterologous transgene.

52. The method of claim 51, wherein the heterologous transgene is operably linked to a promoter.

53. The method of claim 52, wherein the promoter is a constitutive promoter, a tissue-specific promoter, or an inducible promoter.

54. A method of determining the innate immunogenicity to a lipid nanoparticle (LNP) in an individual, the method comprising
a) incubating monocytes from the individual in dendritic cell culture media under conditions in which the monocytes differentiate into dendritic cells,
b) incubating the dendritic cells with the LNP,
c) analyzing the dendritic cells for altered expression of one or more cytokines compared to a suitable control, whereby altered expression of the one or more cytokines produces a cytokine signature,
wherein expression of the cytokine signature following incubation with the LNP indicates innate immunogenicity to the non-viral gene therapy agent in the individual.

55. The method of claim 54, wherein the cytokine signature comprises increased expression of one or more of IP10, MIP1b, CXCL9, and IL2.

56. The method of claim 54, wherein the cytokine signature comprises increased expression of IP10, MIP1b, CXCL9, and IL2.

57. A method of determining a cytokine signature of a gene therapy agent comprising
a) incubating one or more innate immune cell from one or more individuals with the gene therapy agent,
b) analyzing the one or more innate immune cells for altered expression of one or more cytokines compared to a suitable control,
wherein the altered expression of one or more cytokines in step b) indicates the cytokine signature of the gene therapy agent.

58. The method of claim 57, wherein the innate immune cell is a dendritic cell, a monocyte, a macrophage or a natural killer (NK) cell.

59. The method of claim 57 or 58, wherein the one or more innate immune cells are isolated from peripheral blood mononuclear cells from the individual.

60. The method of any one of claims 57-59, wherein the innate immune cell is a dendritic cell.

61. The method of claim 60, wherein the dendritic cell is derived from a monocyte of the one or more individuals.

62. The method of claim 60 or 61, further comprising isolating monocytes from the one or more individuals and incubating the monocytes in dendritic cell culture media to derive dendritic cells from the monocytes prior to incubating the dendritic cells with the gene therapy agent.

63. The method of claim 62 wherein the monocytes are CD14+ monocytes.

64. The method of claim 63 or 63, wherein the monocytes are incubated with the dendritic cell culture media for about 5 to about 10 days or about 7 to about 8 days to derive dendritic cells from the monocytes.

65. The method of any one of claims 57-64, wherein the innate immune cells are replated prior to the incubation with the gene therapy agent of step b).

66. The method of any one of claims 57-65, wherein the gene therapy agent is a viral vector and wherein the innate immune cells are incubated with the gene therapy agent at an MOI of about 1×10³ to about 1×10⁵ or about 1×10⁴.

67. The method of any one of claims 57-65, wherein the gene therapy agent is a non-viral vector, and wherein the innate immune cells are incubated with the non-viral vector at a concentration of about 1 ng/mL to about 1 mg/mL.

68. The method of any one of claims 57-67, wherein the innate immune cells are incubated with the gene therapy agent for about 12 hours to about 36 hours or about 24 hours.

69. The method of any one of claims 57-68, wherein expression of the cytokines in the cytokine signature is increased compared to a suitable control.

70. The method of claim 69, wherein the suitable control is the expression of the cytokines in the cytokine signature from innate immune cells that are not incubated with the gene therapy agent or wherein the suitable control is expression of the cytokines in the cytokine signature from innate immune cells prior to incubation with the gene therapy agent.

71. The method of any one of claims 57-65 and 68-70, wherein the gene therapy agent is a viral vector.

72. The method of claim 66 or 71, wherein the viral vector is an AAV particle.

73. The method of claim 72, wherein the AAV particle comprises an AAV1 capsid, an AAV2 capsid, an AAV3 capsid, an AAV4 capsid, an AAV5 capsid, an AAV6 capsid, an AAV7 capsid, an AAV8 capsid, an AAVrh8 capsid, an AAV9 capsid, an AAV10 capsid, an AAVrh10 capsid, an AAV11 capsid, an AAV12 capsid, an AAVrh32.33 capsid, An AAV-XL32 capsid, an AAV-XL32.1 capsid, an AAV LKO3 capsid, an AAV2R471A capsid, an AAV2/2-7m8 capsid, an AAV DJ capsid, an AAV DJ8 capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid rAAV2/HBoV1 (chimeric AAV/human bocavirus virus 1), an AAV2HBKO capsid, an AAVPHP.B capsid or an AAVPHP.eB capsid, or a functional variant thereof.

74. The method of claim 73, wherein the AAV capsid comprises a tyrosine mutation, a heparin binding mutation, or an HBKO mutation.

75. The method of any one of claims 72-74, wherein the AAV viral particle comprises an AAV genome comprising one or more inverted terminal repeats (ITRs), wherein the one or more ITRs is an AAV1 ITR, an AAV2 ITR, an AAV3 ITR, an AAV4 ITR, an AAV5 ITR, an AAV6 ITR, an AAV7 ITR, an AAV8 ITR, an AAVrh8 ITR, an AAV9 ITR, an AAV10 ITR, an AAVrh10 ITR, an AAV11 ITR, or an AAV12 ITR.

76. The method of claim 75, wherein the one or more ITRs and the capsid of the AAV particle are derived from the same AAV serotype.

77. The method of claim 75, wherein the one or more ITRs and the capsid of the AAV particles are derived from different AAV serotypes.

78. The method of claim 66 or 71, wherein the viral vector is an adenoviral particle.

79. The method of claim 78, wherein the adenoviral particle comprises an capsid from Adenovirus serotype 2, 1, 5, 6, 19, 3, 11, 7, 14, 16, 21, 12, 18, 31, 8, 9, 10, 13, 15, 17, 19, 20, 22, 23, 24-30, 37, 40, 41, AdHu2, AdHu 3, AdHu4, AdHu24, AdHu26, AdHu34, AdHu35, AdHu36, AdHu37, AdHu41, AdHu48, AdHu49, AdHu50, AdC6, AdC7, AdC69, bovine Ad type 3, canine Ad type 2, ovine Ad, or porcine Ad type 3, or a functional variant thereof.

80. The method of claim 66 or 71, where the viral vector is a lentiviral particle.

81. The method of claim 80, wherein the recombinant lentiviral particle is pseudotyped with vesicular stomatitis virus (VSV), lymphocytic choriomeningitis virus (LCMV), Ross river virus (RRV), Ebola virus, Marburg virus, Mokala virus, Rabies virus, RD114, or a functional variant thereof.

82. The method of claim 66 or 71, where the viral vector is a Herpes simplex virus (HSV) particle.

83. The method of claim 82, wherein the HSV particle is an HSV-1 particle or an HSV-2 particle, or a functional variant thereof.

84. The method of any one of claims 57-65 and 67-70, wherein the gene therapy agent is a lipid nanoparticle.

85. The method of any one of claims 67-84, wherein the gene therapy agent comprises nucleic acid encoding a heterologous transgene.

86. The method of claim 85, wherein the heterologous transgene is operably linked to a promoter.

87. The method of claim 86, wherein the promoter is a constitutive promoter, a tissue-specific promoter, or an inducible promoter.

88. A kit for use in the methods of any one of claims 1-87.


SEQUENCE LISTING (1 sequence)

SEQ ID NO: 1 - DNA, 78 bp, synthetic construct
  source 1..78: mol_type = other DNA
cactccctctctgcgcgctcgctcgctcactgaggccgggcgaccaaaggtcgcccacgcccgggctttgcccgggcg